(19) United States
(12) Patent Application Publication
(10) Pub. No.: US 2025/0361538 A1
(43) Pub. Date: Nov. 27, 2025

(54) BACTERIUM, MICROBIAL CONSORTIUM COMPRISING THE SAME AND USES THEREOF

(71) Applicants: THE STATE OF ISRAEL, MINISTRY OF AGRICULTURE & RURAL DEVELOPMENT, AGRICULTURAL RESEARCH ORGANIZATION, Rishon Lezion (IL); ISRAEL OCEANOGRAPHIC & LIMNOLOGICAL RESEARCH LTD., Haifa (IL); B.G. NEGEV TECHNOLOGIES AND APPLICATIONS LTD., AT BEN-GURION UNIVERSITY, Beer Sheva (IL)

(72) Inventors: Lior GUTTMAN, Eilat (IL); Matan MASASA, Eilat (IL); Ariel KUSHMARO, Beer Yaakov (IL)

(73) Assignees: THE STATE OF ISRAEL, MINISTRY OF AGRICULTURE & RURAL DEVELOPMENT, AGRICULTURAL RESEARCH ORGANIZATION, Rishon Lezion (IL); ISRAEL OCEANOGRAPHIC & LIMNOLOGICAL RESEARCH LTD., Haifa (IL); B.G. NEGEV TECHNOLOGIES AND APPLICATIONS LTD., AT BEN-GURION UNIVERSITY, Beer Sheva (IL)

(21) Appl. No.: 18/846,839

(22) PCT Filed: Mar. 15, 2023
(86) PCT No.: PCT/IL2023/050274

§ 371 Date: Sep. 13, 2024

Related U.S. Application Data
(60) Provisional application No. 63/269,362, filed on Mar. 15, 2022.

(51) Int. Cl. C12P 19/04; C12N 9/28; C12P 19/14

(52) U.S. Cl. CPC C12P 19/04; C12N 9/2417; C12P 19/14; C12Y 302/01001

(57) ABSTRACT

The present disclosure provides marine-derived bacterium, consortium comprising the same and uses therefore for hydrolyzing carbohydrate.

### CROSS-REFERENCE TO RELATED APPLICATIONS

This application is a U.S. National Phase of International Patent Application No. PCT/IL2023/050274, filed on Mar. 15, 2023, which claims priority to U.S. Application No. 63/269,362, filed Mar. 15, 2022. The entire contents of these applications are incorporated herein by reference in their entirety.

### SEQUENCE LISTING

The instant application contains a Sequence Listing which has been submitted electronically in XML format is hereby incorporated by reference in its entirety. Said XML copy, created on Jul. 15, 2025, is named 269002_000023_Seq_Listing and is 4,478,711 bytes in size.

### TECHNOLOGICAL FIELD

The present invention relates to bacterium, microbial consortium comprising the same and uses thereof.

### BACKGROUND ART

References considered to be relevant as background to the presently disclosed subject matter are listed below:
Baghel, Ravi S., et al. “Biorefining of marine macroalgal biomass for production of biofuel and commodity chemicals.” Green Chemistry 17.4 (2015): 2436-2443.
Korzen, Leor, et al. “Marine integrated culture of carbohydrate rich Ulva rigida for enhanced production of bioethanol.” RSC Advances 5.73 (2015): 59251-59256.
Naidu, M. A., and P. Saranraj. “Bacterial amylase: a review.” Int J Pharm Biol Arch 4.2 (2013): 274-87.
Arahal, David R., and Antonio Ventosa. “Moderately halophilic and halotolerant species of Bacillus and related genera.” Applications and systematics of Bacillus and relatives (2002): 83-99.
Kumar, Sumit, et al. “Halophiles as a source of polyextremophilic α-amylase for industrial applications.” AIMS Microbiology 2.1 (2016): 1-26.
Park, Byung H., et al. “CAZymes Analysis Toolkit (CAT): web service for searching and analyzing carbohydrate-active enzymes in a newly sequenced organism using CAZy database.” Glycobiology 20.12 (2010): 1574-1584.
Trincone, Antonio. “Update on marine carbohydrate hydrolyzing enzymes: biotechnological applications.” Molecules 23.4 (2018): 901.
Trivedi, Nitin, et al. “An integrated process for the extraction of fuel and chemicals from marine macroalgal biomass.” Scientific reports 6.1 (2016): 1-8.

Acknowledgement of the above references herein is not to be inferred as meaning that these are in any way relevant to the patentability of the presently disclosed subject matter.

### BACKGROUND

Polysaccharides such as starch have various industrial applications as they can be hydrolyzed to monosaccharides by amylases and glucoamylases. These enzymes consist of many sub-families with diverse functional specifications as the optimal activation temperature or salinity, substrate characteristics, etc.

Seaweeds like Gracilaria conferta, Laminaria digitate and Ulva rigida were recognized for their potential as raw biomass for biorefinery for feeds, pharmaceutics and cosmetics, as well as for bioethanol production (Baghel, Ravi S., et al.). This is also due to the relatively high content of starch in the dry biomass of some of these species (e.g., Ulva rigida), up to 31.5% of the dry material (Korzen, Leor, et al.).

Among the starch degrading bacteria, several species of the genus Bacillus have been recommended for the purification of thermostable α-amylases (Arahal, David R).

### GENERAL DESCRIPTION

The present disclosure revealed a new halophilic, halotolerant, heterotrophic bacterial strain of Alkalihalobacillus sp., that consist various carbohydrate-active enzymes (CAZymes) in its genome including those of α-amylases that enables the bacterium to degraded starch through a potential complete metabolism of this polysaccharide into D-glucose monosaccharide and its active nucleotide form of UDP-glucose. Genomic analyses based on 16S rRNA gene and the whole genome content suggest this bacterium, from the gut of a sea urchin, as a novel bacterium of the genus Alkalihalobacillus.

The present disclosure provides in accordance with some aspects, a marine-derived halotolerant bacterium comprising various CAZymes in its genome.

The present disclosure provides in accordance with some aspects, a marine-derived halotolerant bacterium produces various CAZymes.

The present disclosure provides in accordance with some other aspects, a marine-derived halotolerant bacterium being capable of hydrolyzing at least one carbohydrate.

The present disclosure provides in accordance with some other aspects, a microbial consortium comprising at least one marine-derived halotolerant bacterium or any isolate, mutant, spores, enzymes or extracts thereof and/or a conditioned culture medium of the at least one bacterium and/or secreted compounds from the at least one bacterium, wherein the at least one marine-derived halotolerant bacterium comprises various CAZymes in its genome.

The present disclosure provides in accordance with some other aspects, a microbial consortium comprising at least one marine-derived halotolerant bacterium or any isolate, mutant, spores, enzymes or extracts thereof and/or a conditioned culture medium of the at least one bacterium and/or secreted compounds from the at least one bacterium, wherein the at least one marine-derived halotolerant bacterium produces various CAZymes.

The present disclosure provides in accordance with some other aspects, a microbial consortium comprising at least one marine-derived halotolerant bacterium or any isolate, mutant, spores, enzymes or extracts thereof and/or a conditioned culture medium of the at least one bacterium and/or secreted compounds from the at least one bacterium, wherein said at least one bacterium is capable of hydrolyzing at least one carbohydrate.

The present disclosure provides in accordance with some further aspects, a bacterial culture comprising a biomass composition and a bacterium or a microbial consortium, the microbial consortium comprising the at least one marine-derived halotolerant bacterium or any isolate, mutant, spores, enzymes or extracts thereof and/or a conditioned culture medium of the at least one bacterium and/or secreted compounds from the at least one bacterium, wherein the bacterium is characterized by at least one of (i) comprises various CAZymes in its genome, (ii) produces various CAZymes, (iii) capable of hydrolyzing at least one carbohydrate.

The present disclosure provides in accordance with yet some aspects, a method of hydrolyzing carbohydrate, the method comprising contacting a biomass, biomass derivatives or compositions comprising biomass or any derivative thereof with a bacterium or a microbial consortium, wherein the microbial consortium comprising the at least one marine-derived halotolerant bacterium or any isolate, mutant, spores, enzymes or extracts thereof and/or a conditioned culture medium of the at least one bacterium and/or secreted compounds from the at least one bacterium, wherein the bacterium is characterized by at least one of (i) comprises various CAZymes in its genome, (ii) produces various CAZymes, (iii) capable of hydrolyzing at least one carbohydrate.

The present disclosure provides in accordance with yet some aspects, a method of production of bioenergy products or metabolites comprising contacting a biomass or biomass derivatives with a bacterium or a microbial consortium, wherein the microbial consortium comprising the at least one marine-derived halotolerant bacterium or any isolate, mutant, spores, enzymes or extracts thereof and/or a conditioned culture medium of the at least one bacterium and/or secreted compounds from the at least one bacterium, wherein the bacterium is characterized by at least one of (i) comprises various CAZymes in its genome, (ii) produces various CAZymes, (iii) capable of hydrolyzing at least one carbohydrate.

The present disclosure provides in accordance with yet other aspects, a method of identification or isolating at least one bacterium, the method comprising: (a) subjecting a sample comprising uncharacterized bacteria to a high salinity condition and/or high temperature conditions to obtain a treated sample; and b) selecting at least one bacterium from the treated sample, wherein the selected bacterium is capable of hydrolyzing at least carbohydrate.

### EMBODIMENTS

Some embodiments of this disclosure will now be described in the following numbered paragraph. The following description intends to add on the above general description and not limit it in any manner.

1. A marine-derived halotolerant bacterium characterized by at least one of (i) comprises various CAZymes in its genome, (ii) produces various CAZymes, (iii) capable of hydrolyzing at least one carbohydrate.
2. A microbial consortium comprising at least one marine-derived halotolerant bacterium or any isolate, mutant, spores, enzymes or extracts thereof and/or a conditioned culture medium of the at least one bacterium and/or secreted compounds from the at least one bacterium, wherein said at least one bacterium is of Embodiment No. 1.
3. The bacterium of Embodiment No. 1 or the microbial consortium of Embodiment No. 2, wherein said bacterium is present at a low abundance in intestine of a marine urchin.
4. The bacterium or the microbial consortium of Embodiment No. 3, wherein said bacterium is present in Tripneustes gratilla elatensis.
5. The bacterium or the microbial consortium of any one of Embodiments No. 1 to 4, wherein said bacterium is capable of growing in a solution comprising about 3% saline.
6. The bacterium or the microbial consortium of any one of Embodiments No. 1 to 4, wherein said bacterium is capable of growing at 30° C.
7. The bacterium or the microbial consortium of any one of Embodiments No. 1 to 4, wherein said bacterium is capable of growing at 30° C. in a solution comprising about 3% saline.
8. The bacterium or the microbial consortium of any one of Embodiments No. 1 to 7, wherein the carbohydrate is a polysaccharide.
9. The bacterium or the microbial consortium of Embodiment No. 8, wherein said polysaccharide is at least one of (i) cellulose, (ii) starch, (iii) glycogen or (iv) any combination thereof.
10. The bacterium or the microbial consortium of any one of Embodiment Nos. 1 to 7, wherein said carbohydrate is at least one of amylose and amylopectin.
11. The bacterium or the microbial consortium of any one of Embodiment Nos. 1 to 7, wherein said carbohydrate is from an algal source.
12. The bacterium or the microbial consortium of any one of Embodiment Nos. 1 to 7, wherein said carbohydrate is an algal polysaccharide.
13. The bacterium or the microbial consortium of any one of Embodiment Nos. 1 to 7, wherein said carbohydrate is an algal extract.
14. The bacterium or the microbial consortium of any one of Embodiment Nos. 1 to 7, wherein said carbohydrate is an algal biomass.
15. The bacterium or the microbial consortium of any one of Embodiment Nos. 1 to 7, wherein said carbohydrate is one or more of carrageenan, agar, cellulose, alginate, laminarin, fucoidan, ulvan, chitin, starch, xylan, rhamnan sulfate, chrysolaminarin, or any combination thereof.
16. The bacterium or the microbial consortium of any one of Embodiment Nos. 1 to 15, wherein said bacterium expresses genes capable of metabolizing (i) amino acid and derivatives, (ii) carbohydrate, (iii) protein metabolism or (iv) a combination thereof.
17. The bacterium or the microbial consortium of any one of Embodiment Nos. 1 to 15, wherein said bacterium express gene copies of at least one carbohydrate active enzyme (CAZome).
18. The bacterium or the microbial consortium of Embodiment No. 17, wherein said bacterium comprises at least 80 gene copies, optionally at least 90 gene copies of CAZymes.
19. The bacterium or the microbial consortium of any one of Embodiment Nos. 1 to 15, wherein said bacterium contain at least one carbohydrate active enzyme (CAZome).
20. The bacterium or the microbial consortium of any one of Embodiment Nos. 1 to 15, wherein said bacterium produces at least one carbohydrate active enzyme (CAZome).
21. The bacterium or the microbial consortium of any one of Embodiment Nos. 17 to 20, wherein said CAZome comprise at least one glycoside hydrolase (GH) enzyme, at least one glycosyltransferase (GT) enzyme, at least one polysaccharide lyases (PLs) enzyme, at least one carbohydrate esterases (CEs) enzyme, at least one carbohydrate-binding modules (CBM) enzyme, at least one auxiliary activities (AAs) enzyme or any combination thereof.
22. The bacterium or the microbial consortium of Embodiment No. 21, wherein the at least one bacterium contains gene copies of at least one CE enzyme.
23. The bacterium or the microbial consortium of Embodiment No. 21, wherein the at least one bacterium contains gene copies of at least one CBM enzyme.
24. The bacterium or the microbial consortium of Embodiment No. 21, wherein the at least one bacterium contains gene copies of at least one AA enzyme.
25. The bacterium or the microbial consortium of Embodiment No. 21, wherein the at least one bacterium contains gene copies of at least one PL enzyme.
26. The bacterium or the microbial consortium of Embodiment No. 21, wherein the at least one bacterium contains gene copies of at least one GT enzyme.
27. The bacterium or the microbial consortium of Embodiment No. 26, wherein the at least one GT enzyme is at least one of (i) GT4, (ii) GT2, (iii) a combination thereof.
28. The bacterium or the microbial consortium of Embodiment No. 27, wherein said bacterium contain at least 12 GT4 gene copies, optionally at least 15 GT4 gene copies.
29. The bacterium or the microbial consortium of Embodiment No. 27, wherein said bacterium contain at least 5 GT2 gene copies, optionally at least 8 GT2 gene copies.
30. The bacterium or the microbial consortium of Embodiment No. 21, wherein the at least one bacterium contains gene copies of at least one GH enzyme.
31. The bacterium or the microbial consortium of Embodiment No. 30, wherein said GH enzyme is GH4.
32. The bacterium or the microbial consortium of Embodiment No. 30, wherein said GH enzyme is at least one of (i) GH13, (ii) GH32, (iii) GH31, (iv) GH16 or (v) a combination thereof.
33. The bacterium or the microbial consortium of Embodiment No. 32, wherein said bacterium contain at least 6 GH13 gene copies, optionally at least 8 GH3 gene copies.
34. The bacterium or the microbial consortium of Embodiment No. 33, wherein the GH13 is at least one of GH13_31, GH13_29, GH13_18, GH13_14, GH13_5 or a combination thereof.
35. The bacterium or the microbial consortium of Embodiment No. 32, wherein said bacterium contain at least 6 GH32 gene copies, optionally at least 8 GH32 gene copies.
36. The bacterium or the microbial consortium of any one of Embodiment Nos. 1 to 35, wherein said bacterium contain at least one enzyme listed in Table 3.
37. The bacterium or the microbial consortium of any one of Embodiment Nos. 1 to 36, wherein said bacterium is capable of hydrolyzing a carbohydrate into at least one of (i) amino sugars and nucleotide sugars, (ii) D-glucose, (iii) D-glucose-6P or (iv) combination thereof.
38. The bacterium or the microbial consortium of any one of Embodiment Nos. 1 to 37, wherein said bacterium has at least 85% identity with SEQ ID NO:1.
39. The bacterium or the microbial consortium of any one of Embodiment Nos. 1 to 38, wherein said bacterium is from the genus Alkalihalobacillus.
40. The bacterium or the microbial consortium of any one of Embodiment Nos. 1 to 39, wherein said bacterium has at least 85% identity with SEQ ID NO:2.
41. The bacterium or the microbial consortium of any one of Embodiment Nos. 1 to 40, wherein said bacterium has a sequence as provided in SEQ ID NO:2.
42. A bacterial culture comprising a biomass composition and a bacterium or a microbial consortium of any one of Embodiment Nos. 1 to 41.
43. The bacterial culture of Embodiment No. 42, wherein said biomass is an algal biomass.
44. A method of hydrolyzing carbohydrate, the method comprising contacting a biomass, biomass derivatives or compositions comprising biomass or any derivative thereof with a bacterium or a microbial consortium of any one of Embodiment Nos. 1 to 41 with a biomass.
45. The method of Embodiment No. 44, wherein said biomass is an algal biomass or any extract thereof.
46. A method of production of bioenergy products or metabolites comprising contacting a biomass or biomass derivatives with the bacterium or the microbial consortium of Embodiment Nos. 1 to 41.
47. The method according to Embodiment No. 46, wherein the biomass is an organic matter.
48. The method of Embodiment No. 47, wherein said biomass is an algal biomass.
49. A method of identification or isolating at least one bacterium, the method comprising: (a) subjecting a sample comprising uncharacterized bacteria to a high salinity condition and/or high temperature conditions to obtain a treated sample; and b) selecting at least one bacterium from the treated sample, wherein said selected bacterium is capable of hydrolyzing at least carbohydrate.
50. The method of Embodiment No. 49, wherein the sample comprises tissue samples from intestine of a marine urchin.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1F is a graphical overview of an exemplary workflow including the following steps: FIG. 1A feeding sea urchins with a mono-specific algal diet of Ulva fasciata for 8 weeks; FIG. 1B harvesting the whole digestive tract of the animals; FIG. 1C enrichment of the heterotrophic bacterial community in the crude gut samples through incubation in marine broth media; FIG. 1D isolation of bacterial colonies on agar plates with marine broth media; FIG. 1E and FIG. 1F polysaccharide lyases (polylyases) activity assay on isolated bacterial colonies performed in agar plates with mineral salts media and Ulva-polysaccharides extract and in agar control, respectively.

FIG. 2 shows Alkalihalobacillus sp. 16S rDNA sequences genomic repeats alignment.

FIG. 3 is a circular map of the genome of Alkalihalobacillus sp. generated using CGView Comparison Tool, circles (from outside) representing the following: 1. sequence features; 2. GC content; and 3. GC skew, the different colors identify genome content as CDSs (blue); tRNAs (black); rRNAs (red); tmRNAs (pink); and GC Sqews+(green) or −(purple).

FIG. 4 is an evolutionary tree based on comparative analysis of the 16s rRNA gene sequence of the isolated bacterium Alkohalobacillus sp. and phylogenetically close relative bacteria in the database, the evolutionary distances were computed using the Maximum Composite Likelihood method, tree was generated using the MEGA X tool according to Neighbor-Joining method, and bootstrap test was performed in 1000 replicates while the provided number next to each branch of the tree identify the percentage of replicate trees in which the associated taxa were clustered together in the bootstrap test.

FIGS. 5A-5D are graphs showing the optimum salinity and temperature for amylase activity; FIG. 5A shows in vitro Alkalihalobacillus sp. growth in different salinities of 0, 1, 2, 3, and 4% (w/v) at 30° C., FIG. 5B shows extracellular α-amylase enzymatic activity of the isolated Alkalihalobacillus sp. along 96 hours in different salinities of 0, 1, 2, 3, and 4% (w/v) at 30° C., FIG. 5C shows in vitro Alkalihalobacillus sp. growth in different temperature of (25° C., 30° C., 37° C.) in salinity of 3% and FIG. 5D shows extracellular α-amylase enzymatic activity of the isolated Alkalihalobacillus sp. in different temperature of (25° C., 30° C., 37° C.) in salinity of 3%; values are mean±SD, n=3, One unit (U) of α-amylase activity is equal to disappearance of 1 mg/min of the iodine binding starch in the assay reaction.

FIGS. 6A-6C show genome analysis of Alkalihalobacillus sp., FIG. 6A is a column bar presents the coverage of gene analysis with the percent of genes that were either clustered (green) or not clustered (blue) into known subsystem categories, FIG. 6B is a pie chart of the functional categories (as subsystems) in the genome of Alkalihalobacillus sp. as analyzed in RAST, colors indicates the subsystem category as in index shown as FIG. 6C, while the number of annotated genes in each category is also indicated in brackets.

FIG. 7 is a pie chart of the content of functional categories in the genome of isolated bacterium Alkalihalobacillus sp., genes were annotated and categorized into Cluster of Orthologous Groups (COG) while the number of orthologue genes in each category is displayed.

FIG. 8 is a Venn diagram presents the results on the annotated genes of Alkalihalobacillus sp. using the three different tools of HMMER (green), DIAMOND (brownish), or Hotpep (pink), only genes that received a similar annotation in at least two of the three tools were considered as ‘truly annotated’.

FIGS. 9A and 9B are comparative heatmap diagram of the content of CAZymes from the genomes of the isolated bacterium Alkalihalobacillus sp. and other bacteria with an available complete genome in the database the map highlights the present and the cumulative number of copies in the genome (dark blue=0 to dark red=5 or above) of CAZymes of different families, comparison of the isolated bacterium was made against several species of Bacillus which are currently used for α-amylases production in various industries as well as against halophilic bacteria that contain hydrolases or polylyases in their genome, bacterial species which were reported for their capability of starch hydrolysis in in vitro experiments are marked in *, similarity analysis based on the content and number of copies of the CAZymes in the different bacteria was performed and demonstrated by a similarity tree on the left side of the map.

FIG. 10 is a comparative heatmap diagram of the content of CAZymes from the family GH13 in the genomes of the isolated bacterium Alkalihalobacillus sp. and other bacteria with an available complete genome in the database, the map highlights the present and the cumulative number of copies in the genome (dark blue=0 to dark red=5 or above) of GH13 CAZymes of different sub-families, comparison of the isolated bacterium was made against several species of Bacillus which are currently used for α-amylases production in various industries as well as against halophilic bacteria that contain hydrolases or polylyases in their genome, bacterial species which were not verified for their capability of starch hydrolysis in vitro are marked in*, similarity analysis based on the content and number of copies of the GH13 CAZymes in the different bacteria

FIG. 11 is a metabolic mapping performed according to the Kyoto Encyclopedia of Genes and Genomes (KEGG) showing a hypothetical pathway for the metabolism of starch by the isolated bacterium Alkalihalubacillus sp. green boxes present the genes (CAZymes) which have been identified in the genome of the isolated bacterium while the clear boxes present other known genes in the referred pathway between each two given compounds (marked in ∘), arrows present the direction of the metabolic function, the highlighted red lines identify pathways for complete metabolism of starch as suggested by the KEGG mapper.

### DETAILED DESCRIPTION OF EMBODIMENTS

The present disclosure is based on the identification of novel bacterium that was isolated from a marine source, specifically from the gut of the collector sea urchin Tripneustes gratilla elatensis.

As shown herein, the bacterium was tolerant to high saline concentration and high temperatures. Specifically, as shown in the Examples, in vitro studies of α-amylase activity at various salinities (0-4%) and temperatures (25-37° C.) showed that the isolated bacterium was tolerant to saline concentration of even up to 4% and to high temperature of even up to 37° C.

Based on genomic analyses of both the whole-genome content of this bacterium and its 16S rRNA gene suggested that the identified isolated bacterium is a new strain of the genus Alkalihalobacillus , referred to here as Alkalihalobacillus sp.

As further shown herein, genomic analysis indicated that the bacterium genome contains a diverse pool of genes for carbohydrate-active enzymes including, inter alia, those of α-amylases, that enabled this bacterium to degrade starch in in vitro assays into monosaccharides of D-glucose 6-phosphate (6P), D-glucose, and its active nucleotide form of UDP-glucose.

Based on the above, it was suggested that the isolated bacteria can be used for decomposition of organic matter such as polysaccharides. Moreover, it was suggested that the marine isolated bacteria and/or enzymes from the marine isolated bacteria can be used in a salt-tolerance mechanism and/or in decomposing seaweed polysaccharides.

Hence, in accordance with some aspects, the present disclosure provides a marine-derived halotolerant bacterium, wherein said at least one bacterium is characterized by at least one of (i) comprises various CAZymes in its genome, (ii) produces various CAZymes, (iii) capable of hydrolyzing at least one carbohydrate.

The bacterium can be part of a consortium. Hence, in accordance with some aspects, the present disclosure provides a microbial consortium comprising at least one marine-derived halotolerant bacterium or any isolate, mutant, spores, enzymes or extracts thereof and/or a conditioned culture medium of the at least one bacterium and/or secreted compounds from the at least one bacterium, wherein said at least one bacterium comprises various CAZymes in its genome.

In accordance with some aspects, the present disclosure provides a microbial consortium comprising at least one marine-derived halotolerant bacterium or any isolate, mutant, spores, enzymes or extracts thereof and/or a conditioned culture medium of the at least one bacterium and/or secreted compounds from the at least one bacterium, wherein said at least one bacterium produces various CAZymes.

In accordance with some aspects, the present disclosure provides a microbial consortium comprising at least one marine-derived halotolerant bacterium or any isolate, mutant, spores, enzymes or extracts thereof and/or a conditioned culture medium of the at least one bacterium and/or secreted compounds from the at least one bacterium, wherein said at least one bacterium is capable of hydrolyzing at least one carbohydrate.

In the following disclosure, when referring to microbial consortium it is to be understood as referring to bacterium, compositions and methods. Thus, whenever providing a feature with reference to the microbial consortium, it is to be understood as defining the same feature with respect to the bacterium, compositions and methods, mutatis mutandis.

The microbial consortium as used herein refers to a mixture/cocktail including at least one of a bacterium, an archaeon, a protozoa, an algae, a fungi or a combination thereof. When referring to at least one bacterium it should be understood as referring to one bacterium species and/or strain as classified under common scientific classification.

In some embodiments, the microbial consortium comprises one or more bacterium provided that at least one bacterium is marine-derived halotolerant bacterium and is capable of hydrolyzing at least one carbohydrate.

In some embodiments, the bacterium being the subject of the present disclosure was identified from the gut (intestine) of a marine sea urchin and thus can be isolated and/or purified, by any known method in the art as also detailed below.

In some examples, the bacterium is present at a low abundance in intestine of a marine urchin. In some other examples, the bacterium is present in Tripneustes gratilla elatensis.

Abundance in the context of the present disclosure refers to a representation of the relative amount (quantity) of a specific bacterium in the intestine of a marine urchin. This amount can be obtained, by any method known in the art. For example, various molecular-based methods are available to characterize and quantitate the intestine of a marine urchin such as traditional clone libraries; direct sequencing using next-generation parallel sequencing technology; denaturing gradient gel electrophoresis and temperature gradient gel electrophoresis; terminal restriction fragment length polymorphism analysis; fluorescent in situ hybridization; and quantitative Polymerase Chain Reaction (PCR). In addition, computational analysis of sequence data including information on isolated bacteria from the intestine of a marine urchin can be used, for example by counting the number of mapped reads to a reference genome. Using computational tools, relative abundance for each microorganism in the microbiome can be determined and is defined as the number of reads mapped to a reference genome divided by the total number of microbial reads within a given microbiome sample and normalized in methods known in the art (e.g. genome size).

The term low-abundance as used herein refer to a bacterium that is present in the intestine of a marine urchin that is below a predetermined standard value/s or cutoff value/s. In some embodiments, low abundance bacterium has an average relative abundance of less than 0.5%, at times less than 0.4%, at times less than 0.3%, at times less than 0.2%, at times even less than 0.1%. In some embodiments, low abundance bacterium has an average relative abundance of between about 0.00001% to about 0.5%, at times between about 0.0001% to about 0.4%, at times between about 0.001% to about 0.3%, at times between about 0.01% to about 0.2%, at times between about 0.01% to about 0.1% at times between about 0.05% to about 0.1%.

The term bacterium used herein refers in accordance with some embodiments, to at least one of an isolated bacterium, a purified bacterium or a recombinant bacterium.

As described herein, the microbial consortium comprises at least one marine-derived bacterium that was shown to tolerate high salinity. Specifically, as shown in Example 2, the in vitro experiments confirmed that the isolated bacterium was capable to grow under salinity of at least 1%, for example, between 1 and 4%, and temperature of at least 25° C., for example between 25 and 37° C.

In some embodiments, the at least one marine-derived bacterium is a halotolerant bacterium.

As used herein, the term halotolerant bacterium refers to a bacterium that can tolerate high-salt conditions suggesting that this bacterium can grow in a wide ranges of salt concentrations.

In some embodiments, the at least one marine-derived bacterium is a halophilic bacterium.

As used herein, the term halophilic bacterium refers to a bacterium that requires high salt for growth.

In some examples, the bacterium is capable of growing in at least about 1% saline, at times at least about 2%, at times at least about 3%, at times at least about 4% saline.

In some examples, the bacterium is capable of growing in between about 1% saline and about 4% saline.

In some examples, the bacterium is capable of growing in about 1% saline, about 2%, about 3%, at times at about 4%.

In some examples, the bacterium is capable of growing for about 6 hours in 1% salinity, at times for about 12 hours, at times for about 18 hours, at times for about 24 hours, at times for about 36 hours, at times for about 48 hours, at times for about 60 hours, at times for about 72 hours, at times for about 84 hours, at times for about 96 hours in 1% salinity.

In some examples, the bacterium is capable of growing for about 6 hours in 2% salinity, at times for about 12 hours, at times for about 18 hours, at times for about 24 hours, at times for about 36 hours, at times for about 48 hours, at times for about 60 hours, at times for about 72 hours, at times for about 84 hours, at times for about 96 hours in 2% salinity.

In some examples, the bacterium is capable of growing for about 6 hours in 3% salinity, at times for about 12 hours, at times for about 18 hours, at times for about 24 hours, at times for about 36 hours, at times for about 48 hours, at times for about 60 hours, at times for about 72 hours, at times for about 84 hours, at times for about 96 hours in 3% salinity.

In some examples, the bacterium is capable of growing for about 6 hours in 4% salinity, at times for about 12 hours, at times for about 18 hours, at times for about 24 hours, at times for about 36 hours, at times for about 48 hours, at times for about 60 hours, at times for about 72 hours, at times for about 84 hours, at times for about 96 hours in 4% salinity.

In some examples, the bacterium is capable of growing at a temperature of 25° C., at times about 30° C., at times about 37° C.

In some examples, the at least bacterium is capable of growing in at least about 1% saline and/or at a temperature of at least about 25° C.

In some examples, the at least bacterium is capable of growing in at least about 2% saline and/or at a temperature of at least about 25° C.

In some examples, the at least bacterium is capable of growing in about 1% saline, at time about 2% saline, at time about 3% saline, at time about 4% saline at a temperature of about 25° C.

In some examples, the at least bacterium is capable of growing in about 1% saline, at time about 2% saline, at time about 3% saline, at time about 4% saline at a temperature of about 30° C.

In some examples, the at least bacterium is capable of growing in about 1% saline, at time about 2% saline, at time about 3% saline, at time about 4% saline at a temperature of about 37° C.

In some examples, the at least bacterium is capable of growing in about 1% saline, at a temperature of about 25° C., at times about 30° C., at times about 37° C.

In some examples, the at least bacterium is capable of growing in about 2% saline, at a temperature of about 25° C., at times about 30° C., at times about 37° C.

In some examples, the at least bacterium is capable of growing in about 3% saline, at a temperature of about 25° C., at times about 30° C., at times about 37° C.

In some examples, the at least bacterium is capable of growing in about 4% saline, at a temperature of about 25° C., at times about 30° C., at times about 37° C.

In some examples, the at least bacterium is capable of growing at salinities of between about 1% and about 4% and temperatures of between about 25° C. and about 37° C.

In some embodiments, the at least bacterium was capable of growing for at least 96 hours in a solution comprising at least about 1% saline, at least about 2% salinity, at least about 3% salinity, at least about 4% salinity. In some embodiments, the at least bacterium was capable of growing for at least 24 hours, at least 48 hours, at least 72 hours in a solution comprising at least about 1% saline, at least about 2% salinity, at least about 3% salinity, at least about 4% salinity.

In some embodiments, the at least bacterium was capable of growing for at least 96 hours in a solution comprising at least about 25° C., at least about 30° C., at least about 37° C. In some embodiments, the at least bacterium was capable of growing for at least 24 hours, at least 48 hours, at least 72 hours in at least about 25° C., at least about 30° C., at least about 37° C.

It should be noted that bacterium growth can be measured by any method known in the art, for example, the in vitro growth measurements described in the Examples below.

As described herein, the bacterium was capable of hydrolyzing carbohydrate. The term carbohydrate as used herein refers to is a biomolecule consisting of carbon (C), hydrogen (H) and oxygen (O) atoms. The term hydrolyzing as used herein refers to breakdown of the carbohydrates into monomeric sugars.

The present disclosure is not limited to a specific carbohydrate and as described herein may be applicable for a variety of carbohydrates.

In some embodiments, the carbohydrate is a polysaccharide.

In some examples, the at least one bacterium is capable of hydrolyzing at least one polysaccharide.

In some embodiments, the polysaccharide is at least one of (i) cellulose, (ii) starch, (iii) glycogen or (iv) any combination thereof.

In some examples, the at least one bacterium is capable of hydrolyzing starch.

As shown in Example 2 below, in vitro assays showed that the isolated bacterium is capable of producing α-amylases as shown in the α-amylase activity. Interestingly, α-amylase activity was observed at salinities of between 1 and 4% and temperatures of between 25° C. and 37° C.

In some embodiments, the at least bacterium was capable of maintaining amylases activity for at least 96 hours in a solution comprising at least about 1% saline, at least about 2% salinity, at least about 3% salinity, at least about 4% salinity. In some embodiments, the at least bacterium was capable of maintaining amylases activity for at least 24 hours, at least 48 hours, at least 72 hours in a solution comprising at least about 1% saline, at least about 2% salinity, at least about 3% salinity, at least about 4% salinity.

In some embodiments, the at least bacterium was capable of maintaining amylases activity for at least 96 hours in a solution comprising at least about 25° C., at least about 30° C., at least about 37° C. In some embodiments, the at least bacterium was capable of maintaining amylases activity for at least 24 hours, at least 48 hours, at least 72 hours in at least about 25° C., at least about 30° C., at least about 37° C.

The amylases activity can be measured by any method known in the art. For example, the amylase activity was determined using the starch-iodine assay Described herein in the examples below.

In some embodiments, the starch is at least one of amylose and amylopectin.

In some examples, the at least one bacterium is capable of hydrolyzing amylose.

In some examples, the at least one bacterium is capable of hydrolyzing amylopectin.

The carbohydrate, optionally being a polysaccharide may be from an algal source.

In some embodiments, the carbohydrate is a seaweed carbohydrate.

In some embodiments, the carbohydrate is an algal carbohydrate.

In some embodiments, the carbohydrate is a seaweed polysaccharide.

In some embodiments, the carbohydrate is an algal polysaccharide.

In some examples, the at least one bacterium is capable of hydrolyzing an algal carbohydrate.

In some examples, the at least one bacterium is capable of hydrolyzing an algal polysaccharide.

The algal polysaccharide refers to at least one polysaccharide derived from a seaweed source, specifically an algal source and can be in the form of an algae extract or algal biomass.

In some examples, the at least one bacterium is capable of hydrolyzing algae extract.

In some examples, the at least one bacterium is capable of hydrolyzing algae biomass.

In some embodiments, the at least one algal polysaccharide is one or more of carrageenan, agar, cellulose, alginate, laminarin, fucoidan, ulvan, chitin, starch, xylan, rhamnan sulfate, chrysolaminarin, or any combination thereof.

In some examples, the at least one bacterium is capable of hydrolyzing at least one of carrageenan, agar, alginate, laminarin, fucoidan, ulvan, chitin, starch, xylan, rhamnan sulfate, chrysolaminarin or any combination thereof.

In some embodiments, the at least one polysaccharide is at least one of carrageenan, agar, laminarin, ulvan or any combination thereof.

In some examples, the at least one bacterium is capable of hydrolyzing at least one of carrageenan, agar, laminarin, ulvan or any combination thereof.

In some embodiments, the at least one polysaccharide is at least one of carrageenan, laminarin, ulvan or any combination thereof.

In some examples, the at least one bacterium is capable of hydrolyzing at least one of arrageenan, laminarin, ulvan or any combination thereof.

In some embodiments, the at least one polysaccharide is at least one of carrageenan, agar, alginate or any combination thereof.

In some examples, the at least one bacterium is capable of hydrolyzing at least one of carrageenan, agar, alginate or any combination thereof.

In some embodiments, the at least one carbohydrate is carrageenan.

In some embodiments, the at least one carbohydrate is agar.

In some embodiments, the at least one carbohydrate is cellulose.

In some embodiments, the at least one carbohydrate is alginate.

In some embodiments, the at least one carbohydrate is laminarin.

In some embodiments, the at least one carbohydrate is fucoidan.

In some embodiments, the at least one carbohydrate is ulvan.

In some embodiments, the at least one carbohydrate is starch.

In some embodiments, the at least one carbohydrate is chitin.

In some embodiments, the at least one carbohydrate is xylan.

In some embodiments, the at least one carbohydrate is rhamnan sulfate.

In some embodiments, the at least one carbohydrate is chrysolaminarin.

As shown in Example 3, analysis of the whole-genome sequence of the at least one bacterium identified functional genes clustered into 25 subsystem categories, including amino acids and derivatives (372 genes), carbohydrates (274), and protein metabolism (236).

In some embodiments, the at least one bacterium expresses genes capable of metabolizing (i) amino acid and derivatives, (ii) carbohydrate, (iii) protein metabolism or (iv) a combination thereof.

As shown in Example 4, the content of carbohydrate-active enzymes (CAZymes) in the genome also referred as the bacterial CAZome showed that the at least one bacterium contained a pool of CAZymes.

In some embodiments, the at least one bacterium contain at least one carbohydrate active enzyme (CAZome).

In some examples, the at least one bacterium produces at least one CAZome.

Carbohydrate active enzymes are typically identified in CAZy that is a database of Carbohydrate-Active enZYmes (CAZymes).

In some examples, the at least one bacterium comprises at least 20 gene copies, at times at least 30 gene copies, at times at least 40 gene copies, at times at least 50 gene copies, at times at least 60 gene copies, at times at least 70 gene copies, at times at least 80 gene copies, at times at least 90 gene copies of CAZymes.

In some examples, the at least one bacterium comprises 20 gene copies, at times 30 gene copies, at times 40 gene copies, at times 50 gene copies, at times 60 gene copies, at times 70 gene copies, at times 80 gene copies, at times 90 gene copies of CAZymes.

In some examples, the at least one bacterium comprises 94 gene copies of CAZymes.

In some embodiments, the CAZome comprise at least one glycoside hydrolase (GH) enzyme, at least one glycosyltransferase (GT) enzyme, at least one polysaccharide lyases (PLs) enzyme, at least one carbohydrate esterases (CEs) enzyme, at least one carbohydrate-binding modules (CBM) enzyme, at least one auxiliary activities (AAs) enzyme or any combination thereof.

In some examples, the at least one bacterium produces at least one of (i) at least one glycoside hydrolase (GH) enzyme, (ii) at least one glycosyltransferase (GT) enzyme, (iii) at least one polysaccharide lyases (PLs) enzyme, (iv) at least one carbohydrate esterases (CEs) enzyme, (v) at least one carbohydrate-binding modules (CBM) enzyme, (vi) at least one auxiliary activities (AAs) enzyme or (vii) any combination thereof.

In some embodiments, the CAZome comprise at least one glycoside hydrolase (GH) enzyme and/or at least one glycosyltransferase (GT) enzyme.

In some examples, the at least one bacterium produces at least one of (i) at least one glycoside hydrolase (GH) enzyme, (ii) at least one glycosyltransferase (GT) enzyme or (iii) any combination thereof.

In some embodiments, the at least one bacterium produces at least one glycosyltransferase (GT) enzyme.

As used herein glycosyltransferase (GT) refers to an enzyme that establish natural glycosidic linkages by catalyzing the transfer of saccharide moieties from an activated nucleotide sugar (also known as the “glycosyl donor”) to a nucleophilic glycosyl acceptor molecule.

In some embodiments, the at least one glycosyltransferase enzyme is from at least one of (i) GT4, (ii) GT2, (iii) a combination thereof.

In some embodiments, the at least one glycosyltransferase enzyme is a GT4 enzyme.

In some embodiments, the at least one GT4 enzyme is at least one of sucrose synthase (EC 2.4.1.13); sucrose-phosphate synthase (EC 2.4.1.14); α-glucosyltransferase (EC 2.4.1.52); lipopolysaccharide N-acetylglucosaminyltransferase (EC 2.4.1.56); phosphatidylinositol α-mannosyltransferase (EC 2.4.1.57); GDP-Man: Man1GlcNAc2-PP-dolichol α-1,3-mannosyltransferase (EC 2.4.1.132); GDP-Man: Man3GlcNAc2-PP-dolichol/Man4GlcNAc2-PP-dolichol α-1,2-mannosyltransferase (EC 2.4.1.131); digalactosyldiacylglycerol synthase (EC 2.4.1.141); 1,2-diacylglycerol 3-glucosyltransferase (EC 2.4.1.157); diglucosyl diacylglycerol synthase (EC 2.4.1.208); trehalose phosphorylase (EC 2.4.1.231); NDP-Glc: α-glucose α-glucosyltransferase/α,α-trehalose synthase (EC 2.4.1.245); GDP-Man: Man2GlcNAc2-PP-dolichol α-1,6-mannosyltransferase (EC 2.4.1.257); UDP-GlcNAc: 2-deoxystreptamine α-N-acetylglucosaminyltransferase (EC 2.4.1.283); UDP-GlcNAc: ribostamycin α-N-acetylglucosaminyltransferase (EC 2.4.1.285); UDP-Gal α-galactosyltransferase (EC 2.4.1.-); UDP-Xyl α-xylosyltransferase (EC 2.4.2.-); UDP-GlcA α-glucuronyltransferase (EC 2.4.1.-); UDP-Glc α-glucosyltransferase (EC 2.4.1.-); UDP-GalNAc: GalNAc-PP-Und α-1,3-N-acetylgalactosaminyltransferase (EC 2.4.1.306); UDP-GalNAc: N,N′-diacetylbacillosaminyl-PP-Und α-1,3-N-acetylgalactosaminyltransferase (EC 2.4.1.290); ADP-dependent α-maltose-1-phosphate synthase (2.4.1.342); [retaining] UDP-GlcNAc: polypeptide α-N-acetylglucosaminyltransferase (EC 2.4.1.-); UDP-GlcNAc: α-N-acetylglucosaminyltransferase (EC 2.4.1.-); GDP-Man: α-1,4-mannosyltransferase (EC 2.4.1.-)

In some examples, the CAZymes of the GT4 family contain at least two gene copies, at times at least three gene copies, at times at least five gene copies, at times at least seven gene copies, at times at least ten gene copies, at times at least 12 gene copies, at times at least 15 gene copies.

In some examples, the CAZymes of the GT4 family contain two gene copies, at times three gene copies, at times five gene copies, at times seven gene copies, at times ten gene copies, at times 12 gene copies, at times 15 gene copies.

In some examples, the CAZymes of the GT4 family contain 16 gene copies.

In some embodiments, the at least one glycosyltransferase enzyme is a GT2 enzyme.

In some embodiments, the at least one GT2 enzyme is at least one of cellulose synthase (EC 2.4.1.12); chitin synthase (EC 2.4.1.16); dolichyl-phosphate β-D-mannosyltransferase (EC 2.4.1.83); dolichyl-phosphate β-glucosyltransferase (EC 2.4.1.117); N-acetylglucosaminyltransferase (EC 2.4.1.-); N-acetylgalactosaminyltransferase (EC 2.4.1.-); hyaluronan synthase (EC 2.4.1.212); chitin oligosaccharide synthase (EC 2.4.1.-); β-1,3-glucan synthase (EC 2.4.1.34); β-1,4-mannan synthase (EC 2.4.1.-); β-mannosylphosphodecaprenol-mannooligosaccharide α-1,6-mannosyltransferase (EC 2.4.1.199); UDP-Galf: rhamnopyranosyl-N-acetylglucosaminyl-PP-decaprenol β-1,4/1,5-galactofuranosyltransferase (EC 2.4.1.287); UDP-Galf: galactofuranosyl-galactofuranosyl-rhamnosyl-N-acetylglucosaminyl-PP-decaprenol β-1,5/1,6-galactofuranosyltransferase (EC 2.4.1.288); dTDP-L-Rha: N-acetylglucosaminyl-PP-decaprenol α-1,3-L-rhamnosyltransferase (EC 2.4.1.289); alternating β-1,3/4-N-acetylmannan synthase (2.4.1.-); UDP-GlcA: N-acetylglucosaminyl-proteoglycan β-1,4-glucuronosyltransferase (EC 2.4.1.225); [inverting] UDP-Glc: glycocin S-β-glucosyltransferase (EC 2.4.1.-); [inverting] UDP-Glc: protein O-β-glucosyltransferase (EC 2.4.1.-).

In some examples, the CAZymes of the GT2 family contain at least two gene copies, at times at least three gene copies, at times at least five gene copies, at times at least seven gene copies, at times at least eight gene copies.

In some examples, the CAZymes of the GT2 family contain two gene copies, at times three gene copies, at times five gene copies, at times seven gene copies, at times eight gene copies.

In some examples, the CAZymes of the GT2 family contain nine gene copies. In some embodiments, the at least one bacterium produces at least one glycoside hydrolase (GH) enzyme.

As used herein glycoside hydrolase (GH) refers to an enzyme that catalyzes the hydrolysis of glycosidic bonds in complex sugars.

In some embodiments, the at least one glycoside hydrolase enzyme is from GH4.

In some embodiments, the at least one GH4 enzyme is at least one of maltose-6-phosphate glucosidase (EC 3.2.1.122); α-glucosidase (EC 3.2.1.20); α-galactosidase (EC 3.2.1.22); 6-phospho-β-glucosidase (EC 3.2.1.86); α-glucuronidase (EC 3.2.1.139); α-galacturonase (EC 3.2.1.67); palatinase (EC 3.2.1.-). In some embodiments, the at least one glycoside hydrolase enzyme is at least one of (i) GH13, (ii) GH32, (iii) GH31, (iv) GH16 or (v) a combination thereof.

In some embodiments, the at least one glycoside hydrolase enzyme is at least one of GH13.

In some examples, the CAZymes of the GH13 family contain at least two gene copies, at times at least three gene copies, at times at least five gene copies, at times at least seven gene copies, at times at least eight gene copies.

In some examples, the CAZymes of the GH13 family contain two gene copies, at times three gene copies, at times five gene copies, at times seven gene copies, at times eight gene copies.

In some examples, the CAZymes of the GH13 family contain nine gene copies.

In some embodiments, the GH13 enzyme is at least one of GH13_31, GH13_29, GH13_18, GH13_14, GH13_5 or a combination thereof.

In some embodiments, the at least one glycoside hydrolase enzyme is at least one of GH32.

In some embodiments, the GH32 enzyme is at least one of invertase (EC 3.2.1.26); endo-inulinase (EC 3.2.1.7); β-2,6-fructan 6-levanbiohydrolase (EC 3.2.1.64); endo-levanase (EC 3.2.1.65); exo-inulinase (EC 3.2.1.80); fructan β-(2,1)-fructosidase/1-exohydrolase (EC 3.2.1.153); fructan β-(2,6)-fructosidase/6-exohydrolase (EC 3.2.1.154); sucrose: sucrose 1-fructosyltransferase (EC 2.4.1.99); fructan: fructan 1-fructosyltransferase (EC 2.4.1.100); sucrose: fructan 6-fructosyltransferase (EC 2.4.1.10); fructan: fructan 6G-fructosyltransferase (EC 2.4.1.243); levan fructosyltransferase (EC 2.4.1.-); [retaining] sucrose: sucrose 6-fructosyltransferase (6-SST) (EC 2.4.1.-); cycloinulo-oligosaccharide fructanotransferase (EC 2.4.1.-).

In some examples, the CAZymes of the GH32 family contain at least two gene copies, at times at least three gene copies, at times at least five gene copies, at times at least seven gene copies, at times at least eight gene copies.

In some examples, the CAZymes of the GH32 family contain two gene copies, at times three gene copies, at times five gene copies, at times seven gene copies, at times eight gene copies.

In some examples, the CAZymes of the GH32 family contain nine gene copies.

In some embodiments, the at least one glycoside hydrolase enzyme is at least one of GH31.

In some embodiments, the at least one glycoside hydrolase enzyme is at least one of GH16.

In some embodiments, the at least bacterium produces at least one of an amylases and/or a glucoamylase.

In some embodiments, the at least bacterium produces at least one of an auxiliary activity enzyme. An auxiliary activity enzyme refers to one or more families of catalytic proteins that are involved in plant cell degradation through an ability to help other enzymes, such as GH, PL enzymes gain access to the carbohydrates comprising the algae cell wall.

In some embodiments, the at least bacterium produces at least one enzyme from the enzyme families listed in Table 3.

In some embodiments, the at least bacterium produces CAZymes as shown in Table 3.

It should be noted that each row in Table 3 may be considered as a separate embodiment of the present disclosure and hence the at least one bacterium in accordance with the present disclosure may produce one or more enzymes in each one of the enzyme family (listed in separate rows) as well as one or more enzymes from each one of the enzymes family.

In some embodiments, the at least one bacterium comprises for each enzyme family listed in Table 3 number of copies±50%, at times±30%, at times±20%, at times±10%, at times±5% as recited in Table 3 for each enzyme family.

In some embodiments, the at least bacterium comprises for each enzyme family listed in Table 3 number of copies recited in Table 3.

In some embodiments, the at least bacterium is capable of modulating each one of the activities listed in Table 3.

As described herein, the at least one bacterium is capable of hydrolyzing at least one carbohydrate.

In some embodiments, the at least one bacterium is capable of hydrolyzing starch/glycogen.

In some embodiments, the at least one bacterium is capable of hydrolyzing starch/glycogen into amino sugars and nucleotide sugars. In some other embodiments, the at least one bacterium is capable of hydrolyzing starch/glycogen into D-glucose. In some other embodiments, the at least one bacterium is capable of hydrolyzing starch/glycogen into D-glucose-6P.

The at least one bacterium was characterized by an Average Nucleotide Identity (ANI) as compared to other known bacteria.

In some embodiments, the at least bacterium has an Average Nucleotide Identity (ANI) of at least 65% with at least one sequence of Bacillus sp. N1-1 CP 046564, Alkalihalobacillus macyae strain DSM 16346 (JMM-4) or Alteribacter populi strain FJAT 45347.

In some embodiments, the at least bacterium has an Average Nucleotide Identity (ANI) of at least 65% with Bacillus sp. N1-1 CP 046564.

Bacillus sp. N1-1 CP 046564 may be denoted by Taxonomy ID: 2682541, or by Accession No.: PRJNA592026, or by GenBank: CP046564.1. The sequence of Bacillus sp. N1-1 CP 046564 was downloaded from: https://www.ncbi.nlm.nih.gov/assembly/GCF_009818105.1

In some embodiments, the at least bacterium has an Average Nucleotide Identity (ANI) of at least 65%, at times at least 70%, at times at least 75% with Alkalihalobacillus macyae DSM 16346.

Alkalihalobacillus macyae strain DSM 16346 (JMM-4) may be denoted by Taxonomy ID: 157733, or by Accession No.: PRJNA286270. The sequence of Alkalihalobacillus macyae DSM 16346 was downloaded from: https://www.ncbi.nlm.nih.gov/assembly/GCF_001039475.1

In some embodiments, the at least bacterium has an Average Nucleotide Identity (ANI) of at least 65% with Alteribacter populi.

Alteribacter populi strain FJAT 45347 may be denoted by Taxonomy ID: 2011011, or by Accession No.: PRJNA390025. The sequence of Alteribacter populi FJAT 45347 was downloaded from: https://www.ncbi.nlm.nih.gov/assembly/GCF_002352765.1

In some embodiments, the at least bacterium has an Average Nucleotide Identity (ANI) of at least 70%, at least 75%, at least 80%, at least 85%, at least 90%, at least 95%, at least 99% with a sequence of Bacillus sp. N1-1 CP 046564.

In some embodiments, the at least bacterium has an Average Nucleotide Identity (ANI) of at least 65%, at least 70%, at least 75% with a sequence of Alkalihalobacillus macyae strain DSM 16346 (JMM-4).

In some embodiments, the at least bacterium has an Average Nucleotide Identity (ANI) of at least 60%, at least 65% with a sequence of Alteribacter populi strain FJAT 45347.

It should be noted that when referring to phylogenetic analysis based on the whole-genomic sequence, the % identity score to other available genomes may be based on coverage rate of less than 100% at times less than about 90%, less than about 80%, less than about 70% and at times about 66% between the two genomes.

The term identity (% identity) as used herein refer to two or more nucleic acid sequences that are the same. In the context of the present disclosure the sequence identity encompasses transcription changes of DNA to RNA, e.g. T and U are considered identical. The identity may exist over a region of a sequence that is considered by those versed in the art as the whole genomic sequence or any coverage rate thereof as well as the variable region of the 16S rRNA.

In some embodiments, the identity exists over the length the 16S rRNA or a portion thereof of the variable region.

In some embodiments, the identity exists over the whole-genomic sequence.

In some embodiments, the identity exists over a coverage rate of less than 100% at times less than about 90%, less than about 80%, less than about 70% and at times about 66% between the two genomes.

The % identity between two or more nucleic acid sequences is determined for the two or more sequences when compared and aligned for maximum correspondence. In the context of the present disclosure, sequences (nucleic acid) as described herein having % identity are considered to have the same function/activity of the original sequence to which identity is calculated to.

The threshold sequence identity may be 85%, at times 86%, at times 87%, at times 88%, at times 89%, at times 90%, at times 91%, at times 92%, at times 93%, at times 94%, at times 95%, at times 96%, at times 97%, at times 98%, at times 99% with each one of the % identity denoted herein constitute a separate embodiment of the invention.

In some embodiments, the at least bacterium has a full genomic sequence that is at least about 60%, at times at least about 65%, at times at least about 70%, at times at least about 75%, at times at least about 80%, at times at least about 85%, at times at least about 90%, at times at least about 95%, at times at least about 99% identical with SEQ ID NO:1 (Alkalihalobacillus hwajinpoensis SW-72).

In some embodiments, the at least bacterium has a full genomic sequence that is about 60%, at times about 65%, at times about 70%, at times about 75%, at times about 80%, at times about 85%, at times about 90%, at times about 95%, at times about 99% identical to SEQ ID NO:1.

In some embodiments, the at least bacterium has a full genomic sequence that is between about 60% and about 99% identical to SEQ ID NO:1, at times between about 65% and about 99%, at times between about 70% and about 99%, at times between about 75% and about 99%, at times between about 80% and about 99%, at times between about 85% and about 99%, at times between about 90% and about 99%, at times between about 95% and about 99% identical to SEQ ID NO:1.

Alkalihalobacillus hwajinpoensis SW-72 may be denoted by Taxonomy ID: 208199.

The 16S rRNA sequence of Alkalihalobacillus hwajinpoensis SW-72 is provided by GenBank accession No. AF541966, denoted herein by SEQ ID NO:1.

In some embodiments, the at least one bacterium is isolated from sea urchin.

In some embodiments, the at least one bacterium is isolated from sea urchin Tripneustes gratilla.

In some embodiments, the at least one bacterium is from the genus Alkalihalobacillus.

Alkalihalobacillus is a genus of gram-positive or gram-variable rod-shaped bacteria in the family Bacillaceae from the order Bacillales.

In some embodiments, the at least one bacterium belongs to the Alkalihalobacillus hwajinpoensis species.

In some embodiments, the at least one bacterium belongs to one or more of Alkalihalobacillus akibai, Alkalihalobacillus alcalophilus, Alkalihalobacillus algicola, Alkalihalobacillus alkalilacus, Alkalihalobacillus alkalinitrilicus, Alkalihalobacillus alkalisediminis, Alkalihalobacillus berkeleyi, Alkalihalobacillus bogoriensis, Alkalihalobacillus caeni, Alkalihalobacillus clausii, Alkalihalobacillus decolorationis, Alkalihalobacillus gibsonii, Alkalihalobacillus halodurans, Alkalihalobacillus hemicellulosilyticus, Alkalihalobacillus hemicentroti, Alkalihalobacillus hunanensis, Alkalihalobacillus hwajinpoensis, Alkalihalobacillus kiskunsagensis, Alkalihalobacillus krulwichiae, Alkalihalobacillus ligniniphilus, Alkalihalobacillus lindianensis, Alkalihalobacillus lonarensis, Alkalihalobacillus macyae, Alkalihalobacillus marmarensis, Alkalihalobacillus miscanthi, Alkalihalobacillus murimartini, Alkalihalobacillus nanhaiisediminis, Alkalihalobacillus oceani, Alkalihalobacillus okhensis, Alkalihalobacillus okuhidensis, Alkalihalobacillus oshimensis, Alkalihalobacillus patagoniensis, Alkalihalobacillus pseudalcaliphilus, Alkalihalobacillus pseudofirmus, Alkalihalobacillus shaceensis, Alkalihalobacillus trypoxylicola, Alkalihalobacillus urbisdiaboli, Alkalihalobacillus wakoensis or Alkalihalobacillus xiaosiensis species.

In some embodiments, the at least bacterium has a full genomic sequence that is at least about 60%, at times at least about 65%, at times at least about 70%, at times at least about 75%, at times at least about 80%, at times at least about 85%, at times at least about 90%, at times at least about 95%, at times at least about 99% identical with SEQ ID NO:2.

In some embodiments, the at least bacterium has a full genomic sequence that is about 60%, at times about 65%, at times about 70%, at times about 75%, at times about 80%, at times about 85%, at times about 90%, at times about 95%, at times about 99% identical to SEQ ID NO:2.

In some embodiments, the at least bacterium has a full genomic sequence that is between about 60% and about 99% identical to SEQ ID NO:2, at times between about 65% and about 99%, at times between about 70% and about 99%, at times between about 75% and about 99%, at times between about 80% and about 99%, at times between about 85% and about 99%, at times between about 90% and about 99%, at times between about 95% and about 99% identical to SEQ ID NO:2.

In some embodiments, the at least one bacterium has a sequence denoted by SEQ ID NO: 2.

In accordance with some other aspects, the present disclosure provides a bacterial culture comprising a biomass composition and a bacterium or a microbial consortium comprising the bacterium, wherein the bacterium is a marine-derived halotolerant bacterium or any isolate, mutant, spores, enzymes or extracts thereof and/or a conditioned culture medium of the at least one bacterium and/or secreted compounds from the at least one bacterium, wherein the bacterium is characterized by one or more of (i) comprises various CAZymes in its genome, (ii) produces various CAZymes, (iii) capable of hydrolyzing at least one carbohydrate.

“Biomass” as used herein means living matter and refers to organic biological matter.

There are diverse sources of biomass including agricultural, forest, waste, as well as algae, for example, wood, crops, seaweed or animal wastes.

Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure.

In some embodiments, the biomass is a wet biomass.

In some embodiments, the biomass is from wood and agricultural products.

In some embodiments, the biomass is from solid waste.

In some embodiments, the biomass is from bacteria and fungi.

In some embodiments, the biomass is from alcohol, such as ethanol.

In some embodiments, the biomass has a high carbohydrate value.

In some embodiments, the biomass comprises carbohydrates that may be hydrolyzed (degraded) by at least one CAZymes.

In some embodiments, the biomass comprises carbohydrates that may be hydrolyzed (degraded) by at least one CAZymes detailed in Table 3.

In some other embodiments, the biomass includes algal biomass that has a high carbohydrate value.

In some further examples, the biomass comprises about 50% carbohydrate, at times about 60%, at times about 70%, at times about 80% carbohydrate (w/w).

In some other examples, the biomass comprises high amounts of polysaccharides.

In some further examples, the biomass comprises about 50% polysaccharides, at times about 60%, at times about 70%, at times about 80% polysaccharides (w/w).

The biomass is in accordance with some examples from an algae source.

In some embodiments, the biomass is from alga.

In some embodiments, the biomass is an algae extract.

In some other embodiments, the biomass is from microalgal.

As appropriated, alga or algae refers to a diverse group of photosynthetic organisms that may be in unicellular forms or multicellular forms. Algae are typically found in a wide range of aquatic and terrestrial habitats, including oceans and freshwater lakes as well as soil.

The biomass can be from various source of algae.

In some examples, the biomass is from green algae, red algae, brown algae or any combination thereof.

In some examples, the biomass is from green algae.

In some other examples, the biomass is from red algae. In some further examples, the biomass is from brown algae.

In some embodiments, the biomass is from Ulva genus. In some embodiments, the biomass is from an Ulva.

As appropriated, ulva refers to a genus of green algae commonly known as sea lettuce that is a multicellular marine macroalgae typically found in shallow water environments such as estuaries, tide pools, and rocky shores.

In some other embodiments, the biomass is from Gracillaria sp.

As appropriated, Gracilaria refers to a genus of red algae, commonly known as “red seaweed,” typically found in marine environments worldwide.

In some embodiments, the biomass is from Gracillaria sp.

In some further embodiments, the biomass is from kelp.

As appropriated, Kelp refers to a type of large brown algae that is found in cold, nutrient-rich marine environments around the world.

In some embodiments, the biomass is from kelp.

In accordance with some other aspects, the present disclosure provides a method of hydrolyzing at least one carbohydrate, the method comprises contacting a biomass, biomass derivatives or compositions comprising biomass or any derivative thereof with a bacterium or a microbial consortium comprising the bacterium, wherein the bacterium is a marine-derived halotolerant bacterium or any isolate, mutant, spores, enzymes or extracts thereof and/or a conditioned culture medium of the at least one bacterium and/or secreted compounds from the at least one bacterium, wherein the bacterium is characterized by one or more of (i) comprises various CAZymes in its genome, (ii) produces various CAZymes, (iii) capable of hydrolyzing at least one carbohydrate.

In some embodiments in which the biomass is an algal biomass or any extract thereof.

In some embodiments, the hydrolysis of polysaccharides by the method provides at least one monosaccharide. As appreciated, monosaccharide called simple sugar, refers to simplest form of sugar and the most basic units (monomers) from which all carbohydrates are built.

In some embodiments, the hydrolysis by the method provides amino sugars and nucleotide sugars.

In some embodiments, the hydrolysis by the method provides maltose, maltotriose, α-dextrins, glucose.

In some embodiments, the methos provides D-glucose or D-glucose-6P.

In some embodiments, the method provides a nutritional improved alga.

In accordance with some other aspects, the present disclosure provides a method of production of bioethanol products or metabolites comprising contacting a biomass, biomass derivatives or compositions comprising biomass or any derivative thereof with a bacterium or a microbial consortium comprising the bacterium, wherein the bacterium is a marine-derived halotolerant bacterium or any isolate, mutant, spores, enzymes or extracts thereof and/or a conditioned culture medium of the at least one bacterium and/or secreted compounds from the at least one bacterium, wherein the bacterium is characterized by one or more of (i) comprises various CAZymes in its genome, (ii) produces various CAZymes, (iii) capable of hydrolyzing at least one carbohydrate.

In accordance with some other aspects, the present disclosure provides a method of production of bioenergy products or metabolites comprising contacting a biomass, biomass derivatives or compositions comprising biomass or any derivative thereof with a bacterium or a microbial consortium comprising the bacterium, wherein the bacterium is a marine-derived halotolerant bacterium or any isolate, mutant, spores, enzymes or extracts thereof and/or a conditioned culture medium of the at least one bacterium and/or secreted compounds from the at least one bacterium, wherein the bacterium is characterized by one or more of (i) comprises various CAZymes in its genome, (ii) produces various CAZymes, (iii) capable of hydrolyzing at least one carbohydrate.

In accordance with some other aspects, the present disclosure provides a method of production of bioethanol, the method comprising contacting a biomass, biomass derivatives or compositions comprising biomass or any derivative thereof with the bacterium or the microbial consortium comprising the bacterium, wherein the bacterium is a marine-derived halotolerant bacterium or any isolate, mutant, spores, enzymes or extracts thereof and/or a conditioned culture medium of the at least one bacterium and/or secreted compounds from the at least one bacterium, wherein the bacterium is characterized by one or more of (i) comprises various CAZymes in its genome, (ii) produces various CAZymes, (iii) capable of hydrolyzing at least one carbohydrate.

As used herein bioethanol also denoted as bioethanol fuel is produced mainly by sugar fermentation process.

The present disclosure also encompasses a bioreactor comprising a bacterial culture.

In accordance with some other aspects, the present disclosure provides a method of identification or isolating at least one bacterium, the method comprising: a) providing a sample comprising uncharacterized bacteria; b) subjecting the sample to a high salinity condition and/or high temperature conditions to obtain a treated sample; and c) selecting at least one bacterium from the treated sample, wherein said selected bacterium is capable of hydrolyzing at least carbohydrate.

In accordance with some aspect, it is provided a method of identification or isolating at least one bacterium, the method comprising: a) subjecting a sample comprising uncharacterized bacteria to a high salinity condition and/or high temperature conditions to obtain a treated sample; and b) selecting at least one bacterium from the treated sample, wherein said selected bacterium is capable of hydrolyzing at least carbohydrate.

In some embodiments, the sample comprises tissue samples from intestine of a marine urchin.

In accordance with some aspect, it is provided a method of identification or isolating at least one bacterium, the method comprising: a) providing a tissue sample from intestine of a marine urchin comprising uncharacterized bacteria; b) subjecting the sample to a high salinity condition and/or high temperature conditions to obtain a treated sample; and c) selecting at least one bacterium from the treated sample, wherein said selected bacterium is capable of hydrolyzing at least carbohydrate.

In accordance with some aspect, it is provided a method of identification or isolating at least one bacterium, the method comprising: a) subjecting a tissue sample from intestine of a marine urchin comprising uncharacterized bacteria to a high salinity condition and/or high temperature conditions to obtain a treated sample; and b) selecting at least one bacterium from the treated sample, wherein said selected bacterium is capable of hydrolyzing at least carbohydrate.

The selection of a bacterium that is capable of hydrolyzing at least carbohydrate can be done by any method capable of monitoring carbohydrate hydrolysis as described herein in the examples.

In some embodiments, the method of the invention for identification or isolating at least one bacterium may be applicable for selecting at least one bacterium capable of hydrolyzing at least one algal carbohydrate, optionally at least one algal polysaccharide.

In some embodiments, the method of the invention for identification or isolating at least one bacterium may be applicable for selecting at least one bacterium capable of hydrolyzing algal polysaccharide.

In some embodiments, the method of the invention for identification or isolating at least one bacterium may be applicable for selecting at least one bacterium capable of hydrolyzing an algal extract.

In some embodiments, the method of the invention for identification or isolating at least one bacterium may be applicable for selecting at least one bacterium capable of hydrolyzing an algal polysaccharide extract.

In some embodiments, the method of the invention for identification or isolating at least one bacterium may be applicable for selecting at least one bacterium capable of hydrolyzing an algal biomass.

In some embodiments, the method of the invention for identification or isolating at least one bacterium may be applicable for selecting at least one bacterium capable of hydrolyzing at least one of carrageenan, agar, cellulose, alginate, laminarin, fucoidan, ulvan, chitin, starch, xylan, rhamnan sulfate, chrysolaminarin, or any combination thereof.

In some embodiments, the method of the invention for identification or isolating at least one bacterium may be applicable for selecting at least one bacterium capable of hydrolyzing at least one of carrageenan, agar, alginate, laminarin, fucoidan, ulvan, xylan, rhamnan sulfate, chrysolaminarin or any combination thereof.

In some embodiments, the method of the invention for identification or isolating at least one bacterium may be applicable for selecting at least one bacterium capable of hydrolyzing an algal biomass, optionally polysaccharide extract, optionally comprising at least one of carrageenan, agar, starch, laminarin, ulvan or any combination thereof.

In some embodiments, the method of the invention for identification or isolating at least one bacterium may be applicable for selecting at least one bacterium capable of hydrolyzing at least one of carrageenan, agar, laminarin, ulvan or any combination thereof.

In some embodiments, the method of the invention for identification or isolating at least one bacterium may be applicable for selecting at least one bacterium capable of hydrolyzing at least one of carrageenan, laminarin, ulvan or any combination thereof.

In some embodiments, the method of the invention for identification or isolating at least one bacterium may be applicable for selecting at least one bacterium capable of hydrolyzing at least one of arrageenan, laminarin, ulvan or any combination thereof.

In accordance with some aspects, the present disclosure provides at least one bacterium capable of hydrolyzing at least carbohydrate obtained by a method comprising a) providing a sample comprising uncharacterized bacteria; b) subjecting the sample to a high salinity condition and/or high temperature conditions to obtain a treated sample; and c) selecting at least one bacterium from the treated sample, wherein said selected bacterium is capable of hydrolyzing at least carbohydrate.

In accordance with some aspects, the present disclosure provides at least one bacterium capable of hydrolyzing at least carbohydrate obtained by a method comprising: a) subjecting a sample comprising uncharacterized bacteria to a high salinity condition and/or high temperature conditions to obtain a treated sample; and b) selecting at least one bacterium from the treated sample, wherein said selected bacterium is capable of hydrolyzing at least carbohydrate.

In accordance with some aspects, the present disclosure provides at least one bacterium capable of hydrolyzing at least carbohydrate obtained by a method comprising: a) providing a tissue sample from intestine of a marine urchin comprising uncharacterized bacteria; b) subjecting the sample to a high salinity condition and/or high temperature conditions to obtain a treated sample; and c) selecting at least one bacterium from the treated sample, wherein said selected bacterium is capable of hydrolyzing at least carbohydrate.

In accordance with some aspects, the present disclosure provides at least one bacterium capable of hydrolyzing at least carbohydrate obtained by a method comprising: a) subjecting a tissue sample from intestine of a marine urchin comprising uncharacterized bacteria to a high salinity condition and/or high temperature conditions to obtain a treated sample; and b) selecting at least one bacterium from the treated sample, wherein said selected bacterium is capable of hydrolyzing at least carbohydrate.

In accordance with some aspects, the present disclosure provides at least one bacterium capable of hydrolyzing at least carbohydrate obtainable by a method comprising a) providing a sample comprising uncharacterized bacteria; b) subjecting the sample to a high salinity condition and/or high temperature conditions to obtain a treated sample; and c) selecting at least one bacterium from the treated sample, wherein said selected bacterium is capable of hydrolyzing at least carbohydrate.

In accordance with some aspects, the present disclosure provides at least one bacterium capable of hydrolyzing at least carbohydrate obtainable by a method comprising: a) subjecting a sample comprising uncharacterized bacteria to a high salinity condition and/or high temperature conditions to obtain a treated sample; and b) selecting at least one bacterium from the treated sample, wherein said selected bacterium is capable of hydrolyzing at least carbohydrate.

In accordance with some aspects, the present disclosure provides at least one bacterium capable of hydrolyzing at least carbohydrate obtainable by a method comprising: a) providing a tissue sample from intestine of a marine urchin comprising uncharacterized bacteria; b) subjecting the sample to a high salinity condition and/or high temperature conditions to obtain a treated sample; and c) selecting at least one bacterium from the treated sample, wherein said selected bacterium is capable of hydrolyzing at least carbohydrate.

In accordance with some aspects, the present disclosure provides at least one bacterium capable of hydrolyzing at least carbohydrate obtainable by a method comprising: a) subjecting a tissue sample from intestine of a marine urchin comprising uncharacterized bacteria to a high salinity condition and/or high temperature conditions to obtain a treated sample; and b) selecting at least one bacterium from the treated sample, wherein said selected bacterium is capable of hydrolyzing at least carbohydrate.

The methods of the present disclosure are applicable to various carbohydrates as described herein.

The following summary may be considered in the context of the present disclosure based on the examples described below.

The two phylogenetic analyses following the whole genome sequencing resulted in some conflict as each allocated isolated bacterium under different taxonomic lineage of either Alkalihalobacillus or Bacillus. This conflict can be attributed to the relatively low coverage during genome comparison with other bacteria in the database as only ⅔ of genome sequences were aligned successfully against other bacteria. Moreover, the two tools that were used for genome comparison differs in their BLAST-algorithm and may contribute to the disagreement in bacterial identification. An uncertainty in the identification of a bacterium as either Bacilli or Alkalihalobacilli seems common as only recently six species of the Bacillus genus were reclassified following a comparative genomic analysis under the new genus taxon of Alkalihalobacillus. In fact, one of these reclassified microbes was Alkalihalobacillus hwajinpoensis SW-72, which has been identified in the current study as bacterial strain with the most similar 16S rRNA gene sequence to that of the Alkalihalobacillus sp. isolate. Beside this genetic similarity, the A. hwajinpoensis SW-72 strain, originally isolated from the East China Sea in Korea, also present other physiological characteristics that are shared with the Alkalihalobacillus sp. isolate. Among, are the optimal growth at temperature between 3° and 35° C., and at salinity between 2 and 5 2-5%. However, while A. hwajinpoensis SW-72 was reported for its activity in decomposing of various carbohydrates as melibiose, D-cellobiose, D-ribose D-galactose, D-mannose, or D-raffinose, no report was found on potential activity of this strain in starch degradation and its CAZome is lack of genes for amylases. It was therefore assumed that the isolated bacterium is a novel halophilic specie of the genus Alkalihalobacillus which is also lacking the ability to grow in a non-salted environment as demonstrated in the present disclosure.

As novel α-amylases are still of interest for industrial uses, the comparative analysis of the bacterial CAZome content encompassed specific members of the Bacillus genus which are currently used for production of commercial α-amylases, as well as several halophilic bacteria that consist of hydrolases or polylyases in their CAZome. Among the compared bacteria were members of the genus Bacillus (i.e. B. subtilis, B. cereus, B. licheniformis, B. amyloliquefaciens and B. paralicheniformis) which presents high commercial value due to their use in the food industry⁴. The comparative analysis of bacterial CAZomes revealed that only one GH was found in all of them (GH23). On the other hand, the interesting differences in GH content can be attributed to different metabolisms of dietary polysaccharides, including starch.

The most diverse CAZyme family in the different bacterial CAZomes was GH13 which contained 21 subfamilies (as shown herein below). While among these, 5 subfamilies (GH5, GH14, GH18, GH29, GH31) were discovered in the genome of Alkalihalobacillus sp., with the unique and highest number of genomic copies found for GH13_14 encodes for pullulanase (EC 3.2.1.41). This enzyme hydrolyzes the α-1,6 glucosidic linkages in starch, amylopectin and pullulan and is essential to complete an efficient conversion of the polysaccharides into small fermentable sugars.

The highest similarity was found between the isolated Alkalihalobacillus sp. and the halophilic Bacillus sp. N1-1 which is also capable of starch degradation. The comparison of representative genera of Bacillus with Alkalihalobacillus sp. revealed that two genes of GH13_29 (α-glucosidase) and GH13_31 (Hexosyltransferases) were shared in the genomes among all Bacillus species and Alkalihalobacillus sp. However, the analyses also highlighted the fact that fundamental knowledge on bacterial CAZymes content could not be used as a sole indicator for enzymatic activity per se. The latter has been evident in the examination of the two strains of Croceivigra radicis which own a similar set of five α-amylases in their genomes but were both found uncapable of starch hydrolysis in vitro¹⁸. In contrary, hydrolysis of starch has been reported in Virgibacillus alimentarius although this bacterium consists of only a single α-amylase of GH13_20 (in four copies) in the genome.

The in vitro experiments confirmed that the isolated bacterium Alkalihalobacillus sp. is capable to grow under salinity of at least 1%, for example, between 1 and 4%, and temperature of at least 25° C., for example between 25 and 37° C. Nevertheless, stability of α-amylases of this bacterium at a more extreme conditions as higher salinity or temperature is considerable. This has been evident in several thermostable α-amylases currently used in the industry which are produced by members of the genus Bacillus of B. subtilis, B. licheniformis, or B. amyloliquefaciens, while these bacteria have been isolated from habitats of a more moderate temperature as the gastrointestinal tract of ruminants, soil or plant roots. Another evidence is from the study of the marine bacterium Pseudoalteromonas neustonica SM1927 which was isolated from habitat of salinity level of 3% but revealed an optimal hydrolysis of starch at much higher salinity between 6-10% in the in vitro trials.

The integration of in vitro experiments for the study of a potential activity in an isolated bacterium together with genomics analyses, as performed in the current research, is of highly important for better understanding of the potential mechanism in which the substrate is metabolized and the related set of genes for the process. This is also since in some cases, an efficient degradation of a substrate into desired chemicals may require the construction of a set of enzymes which may not always derived from a single organism (e.g. bacterium). With this respect, the metabolism of starch in free-living bacteria has been proposed to require the following set of core genes (each in at least one copy) of ADP-glucose pyrophosphorylase (GH13_18), glycogen synthase, glycogen phosphorylase, and branching and debranching enzymes. While consisting all of them in its genome, Alkalihalobacillus sp. was also found to hold few other genes that are related to starch metabolism of families GH13_5, GH13_14, and GH13_31, which are involved in the metabolism of the malto-oligosaccharides of maltodextrin, maltose and dextrin³. The presence of GH13_31 and GH31 in the isolated Alkalihalobacillus sp. is of highly important as these CAZymes are responsible for the consequent metabolism of the respected oligosaccharides dextrin and maltose, into monosaccharides of D-glucose. If not occur, the entire metabolism of starch may be inhibited by the accumulation of either of these oligosaccharides dextrin or maltose. Following this, it was proposed the isolated Alkalihalobacillus sp. to allow a complete metabolism of starch into either D-glucose, D-glucose 6P, or UDP-glucose, with the latter as a precursor for amino sugar and nucleotide sugar metabolism.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

It should be noted that various embodiments of this invention may be presented in a range format. The description of a range should be considered to have specifically disclosed all the possible sub ranges as well as individual numerical values within that range. For example, description of a range such as from 1 to 6 or between 1 and 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

It is to be understood that the terminology used herein is used for the purpose of describing particular embodiments only and not intended to be limiting since the scope of the present invention will be limited only by the appended claims and equivalents thereof.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments unless the embodiment is inoperative without those elements.

It should be noted that the various embodiments and examples detailed herein in connection with various aspects of the invention may be applicable to one or more aspects disclosed herein. It should be further noted that any embodiment described herein, for example, related to components of the food ingredient, may be applied separately or in various combinations. Various embodiments and aspects of the present invention as delineated hereinabove and as claimed in the claims section below find experimental support in the following examples. The phrases “in another embodiment” or any refence made to embodiment as used herein do not necessarily refer to different embodiment, although it may. Thus, various embodiments of the invention can be combined (from the same or from different aspects) without departing from the scope of the invention.

Various embodiments and aspects of the present invention as delineated herein above and as claimed in the claims section below find experimental support in the following examples.

Disclosed and described, it is to be understood that this invention is not limited to the particular examples, bacterium and methods steps, disclosed herein as such methods steps and reactors may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### Some Non-Limiting Examples

### Bacterial Isolation

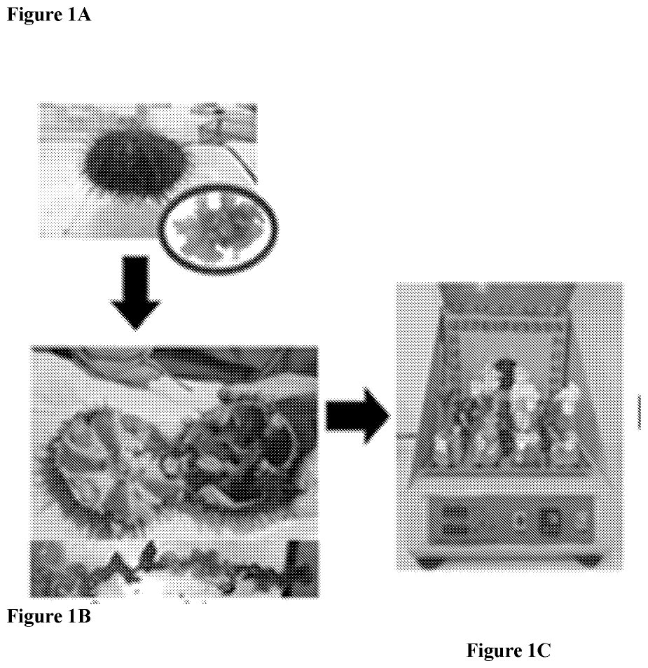

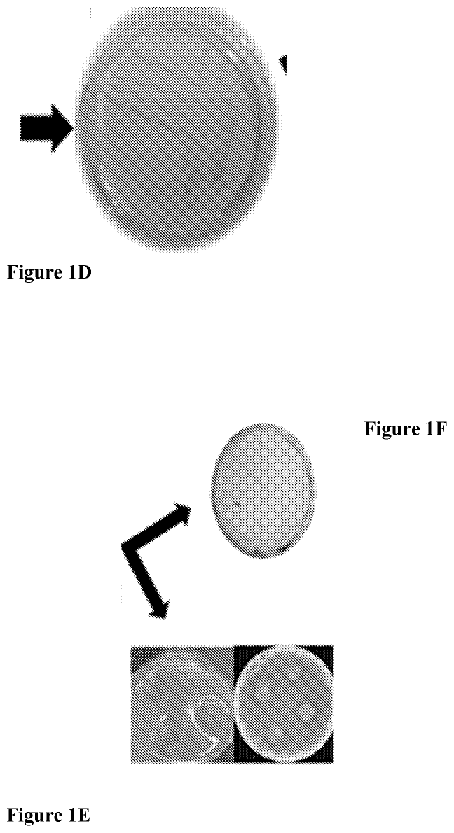

The gut microbiome of sea urchin Tripneustes gratilla was selected for isolation of starch-hydrolyzing bacteria as it contains bacteria which were correlated with starch-rich dietary seaweeds of Ulva or Gracilaria. To enrich the gut with such bacteria, adult sea urchins were fed a mono-specific algal diet of Ulva fasciata during eight weeks prior harvesting of the gut. Enrichment of gut bacteria continued in the lab in flasks with 100 ml marine broth media at salinity of 4% (as in the Red Sea) by incubation at 25° C. as in the echinoculture facility (FIG. 1). To enrich the targeted heterotrophic bacteria, first the content of organic and inorganic residuals was reduced from the crude gut by diluting the culture medium by half every three days along 18 days using a similar bacterial-free medium. Three aliquots of 100 μl each were then sampled from each of the flasks and planted separately on an agar plate (1.5%) with the same medium. Bacterial colonies were transferred to new plates until their isolation. Each of the isolated colonies was transferred to a sterile flask that contained 100 ml of mineral salts liquid medium (4% salinity) and was provided also with a polysaccharides extract from Ulva (Trivedi, Nitin, et al.) as the sole source of carbohydrates at a rate of 2%. After 24 h, 100 μl of the culture was replanted on agar plates with the same Ulva-polysaccharides extract (2%) for 24 h at 37° C., followed by the examination of polylyases activity through the Cetyl pyridinium chloride activity assay. A similar reaction was performed in agar plates of which Ulva-polysaccharides extract was not added as a control (FIG. 1).

### In Vitro Growth and α-Amylase and Glucoamylase Activity Measurements

Bacterial growth and α-amylase activity were measured in cultures at different salinities and temperatures using the recommended liquid media for starch degrading bacteria with 0.2% of commercial starch (Sigma-Aldrich, Israel).

In the first experiment, the isolated Alkalihalobacillus sp. was examined in cultures at different salinities of 0, 1, 2, 3, or 4% (Red Sea salt). A following experiment was performed at different temperatures of 25, 30, and 37° C., all at a salinity level of 3% which was identified in first experiment as favorable for both growth and amylases activity. Both experiments included two sets of control treatment (in triplicates): the first was of starch-free culture medium with the isolated bacterium; and the second was of starch-containing, bacterial-free, culture medium. In both experiments, 1 ml from an enriched culture of isolated Alkalihalobacillus was used as starter. Bacterial growth was measured according cell density at 600 nm while sampling of 1 ml from each culture once every 4 h during the first 48 h of the experiment, and once every 12 h during the following 48 h. Measurement of the exocellular amylases activity was performed at similar regime on similar samples of which the bacterial-free supernatant (100 μl) after centrifugation (10,000 rpm, 15 minutes) was used. The amylases activity assay was the recommended starch-iodine assay in which the bacterial-free supernatant was added to 400 μl of commercial 0.2% starch solution and incubated for 30 min at 50° C., prior stopping the activity with HCl and measuring the remained starch through the Iodine assay and following absorbance at 580 nm.

Statistical analysis of the data on growth or amylases activity was performed using two-way ANOVA and examined the effects of the various factors of time, salinity, or temperature.

### Genomic DNA Extraction, Genome Sequencing and Annotation

Bacterial genomic DNA was purified using the PureLink™ Microbiome Purification Kit following manufacturer protocol. Quality and quantity of genomic DNA were determined in qubit fluorometer and in an Agilent 2100 bioanalyzer. Whole-genome sequencing was performed using a MinION sequencer at the Research Resources Center (University of Illinois, Chicago). The Porechop tool (v0.2.4) was used to trim adapters from resulted sequence while an additional removal of internal adapters was made at identity threshold of 90%. Sequences shorter than 1000 bp were discarded. De novo assembly was performed using the Canu assembler³⁴ with a following error correction using Racon. The Circlator platform³⁶ was used for circularization of the contigs assembly into a complete genome. Illumina MiSeq platform and Nextera XT kit were used for generation of short-read sequences which were assigned together with the long-read sequence to an iterative polishing step of removal of the remained errors as those in repeated region. The Unicycler hybrid assembler (v0.4.1) integrated the data from the Illumina and Oxford Nanopore to produce a complete assembly of a single chromosome. Finally, sequences of contigs were corrected via multiple rounds of mapping read data to contigs by the Burrows-Wheeler Aligner_v0.7.17³⁸.

### Genome-Based Identification and Taxonomic Classification

The 16S rRNA gene seque nce was uploaded to the EzBioCloud server to measure similarity against other sequences in database according Average Nucleotide Identity (ANI), while the all-species Living Tree Project and megablast were used to align this gene with sequences in the SILVA and NCBI databases, respectively. The whole genome sequence was aligned against other prokaryotic genomes in the NCBI database using the Microbial Genomes Atlas (MiGA) while overall genome-related index scores (OGRI) were calculated based on the tetra-nucleotide signatures from another alignment using the JSpeciesWS server and data in GenomesDB.

### Genome Functional Annotation and a Comparative Analysis of the CAZome Content

Genome annotation was performed using the Rapid Annotations using Subsystems Technology (RAST) server and rapid Prokaryotic Genome Annotation software (Prokka), followed by the generation of a genomic map in the CGView server. Orthologous genes were collected, functionally annotated, and clustered in categories using the Webmga and BlastKOALA servers, and their respected databases of Cluster of Orthologous Groups (COG) and Kyoto Encyclopedia of Genes and Genomes (KEGG)⁴⁹. The genomic sequence was scanned for its content of carbohydrate active enzymes (CAZymes) using DbCAN, with a following annotations into enzyme classes of GH, CBM, polysaccharide lyase (PL), glycoside transferase (GT), carbohydrate esterase (CE), or auxiliary activities (AA). Gene annotation was verified only when a similar annotation was identified in at least two of the three databases of HMMER, DIAMOND, or Hotpep. The DbCAN platform was used to analyze the CAZome content of the isolated Alkalihalobacillus sp. and to further compare it against bacterial CAZomes that were extracted from available genomes of some selected bacteria in the BacDive database, which are listed in Table 1.

Table 1 shows a list of the selected halophile bacteria with representative genomes in the NCBI database (including Bio-project number) that were used for CAZymes comparison. The table also indicates the following for each of the bacterial species: if reported capable of starch degradation (including reference) and whether CAZome consist of GHs or PLs. Halophile species are also indicated.

TABLE 1
A list of the selected halophile bacteria

| | | In vitro starch | | |
|---|---|---|---|---|
| | | hydrolysis | | |
| | | (with | | |
| Species: | Bio-project | reference) | Halophile | |
| Croceivirga radicis HSG9 | PRJNA358143 | − | + | Hu et al., 2017 |
| Croceivirga radicis S86 | PRJNA65289 | − | + | Hu et al., 2017 |
| Pseudoalteromonas | PRJDB4787 | − | + | Hwang et al., 2016 |
| neustonica PAMC 28425 | | | | |
| Pseudoalteromonas | PRJNA554250 | − | + | Hwang et al., 2016 |
| neustonica SM1927 | | | | |
| Pseudorhodobacter | PRJNA286976 | − | + | Lee et al., 2016 |
| PAMC27389 | | | | |
| Aureimonas altamirensis | PRJEB18307 | + | + | Jurado et al. 2006 |
| DSM21988 | | | | |
| Aureimonas altamirensis | PRJNA260421 | + | + | Jurado et al. 2006 |
| ON-56566 | | | | |
| Aequorivita sublithincola | PRJNA46631 | − | + | Lucas et al. 2012 |
| DSM14238 | | | | |
| Henriciella algicola | PRJNA381091 | − | + | Rathsack et al. 2006 |
| CCUG67844 | | | | |
| Henriciella barbarensis | PRJNA381091 | − | + | Rathsack et al. 2006 |
| CCUG66934 | | | | |
| Virgibacillus alimentarius | PRJNA238855 | + | + | Kim et al. 2011 |
| J18T | | | | |
| Virgibacillus chiguensis | PRJEB18238 | + | + | Wang et al. 2008 |
| CGMCC1.6496 | | | | |
| Bacillus subtilis | PRJNA76 | + | − | Konsula et al. 2004 |
| Bacillus cereus | PRJNA719349 | + | − | Annamalai et al. |
| | | | | 2011 |
| Bacillus licheniformis | PRJNA293170 | + | − | Saito et al. 1973 |
| Bacillus amyloliquefaciens | PRJNA73591 | + | − | Deb et al. 2013 |
| Bacillus paralicheniformis | PRJNA412146 | + | − | Božić et al. 2020 |
| Halomonas meridiana | PRJNA416452 | + | + | Coronado et al. 2000 |
| Halocella sp. SP 3-1 | PRJNA493542 | + | + | Heng et al. 2019 |
| Planococcus | PRJNA60823 | + | + | Too et al. 2017 |
| donghaensis JHIT | | | | |
| Halothermothrix | PRJNA16377 | + | + | Tan et al. 2008 |
| orenii H168 | | | | |
| Streptomyces | PRJNA189 | + | + | Samiea et al. 2012 |
| avermitilis MA-4680 | | | | |
| Halobacillus halophilus | PRJEA50647 | + | + | Amoozegar et al. 2003 |
| Bacillus sp. N1-1 | PRJNA592026 | + | + | Wang et al. 2020 |
| Alkalihalobacillus sp. | | + | + | |
| | | (current study) | | |

The selected bacteria for this comparison included six Bacillus sp. which are known for their α-amylases production (also commercial ones) and capability of starch hydrolysis. In addition to Bacillus, genomes of several halophilic bacteria were selected and included halophiles that were either reported to be capable of starch hydrolysis in vitro, or that presented genome with either GH or PL CAZymes. Finally, a map of starch metabolism by isolated Alkalihalobacillus sp. was constructed according CAZymes content using the KEGG Mapper.

### Results

### Example 1: Bacterial Isolation and Identification

The enrichment of heterotrophic-halotolerant bacteria resulted in a single bacterium that presented an activity of polylyases when cultured on an assay plate with the Ulva-polysaccharides extract while such an activity was not documented for this bacterium in similar assay plate where Ulva-polysaccharides extract was not added as a control (FIG. 1).

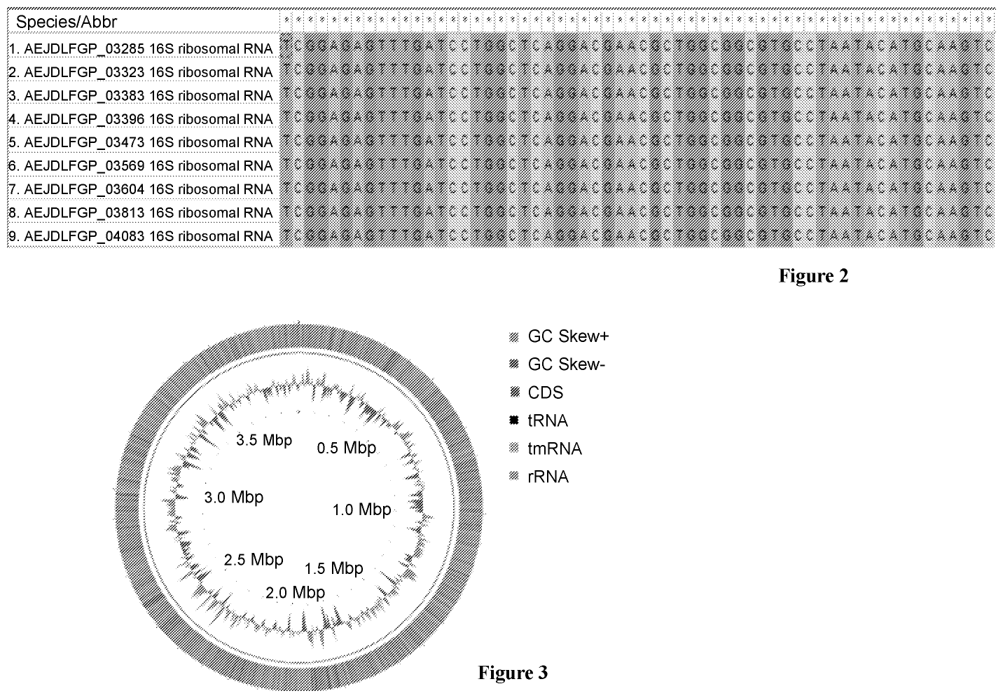

FIG. 2 shows alignment of the intragenomic 16S gene copies in genome using a similarity threshold of >98.65. A full identity (100%) was measured and approved the axenicity of the isolated bacterium.

The whole-genome sequence was generated by combining data from the Illumina and Nanopore sequencing platforms. Analysis of the Nanopore sequencing generated 400Mbp of which 52,900 sequence reads were of high quality with an average read length of 7,561 bp. The resulted, plasmids-free, high-quality contig was at length of 4,461,509 bp in which the mean coverage for a single read was 87.48 and G+C content was 40.4% (data not shown). The iterative polishing step against reads from Illumina sequencing revealed a nearly full identity of the aligned reads (99.89%) and their integration into a single circular chromosome that contained 4,463,966 bp with a mean coverage of 1195.9 (data not shown). Finally, the resulted full genome consisted 4,472 coding sequences (CDS) that were annotated to 90 tRNA and 27 rRNA genes, and an additional single gene of tmRNA (FIG. 3).

Phylogenetic analysis based on the whole-genomic sequence proposed the isolate as a novel bacterial strain with low identity to other available genomes. The most identical bacterial strain in the MiGA database was Bacillus sp. N1-1 CP 046564 with an ANI score of 99.18% but this score was based on coverage rate of only ˜66% between the two genomes. The phylogenetic analysis using available genomes in the JSpeciesWS database revealed low ANI scores of the isolate when compared with Alkalihalobacillus macyae DSM 16346 (77.05%), or Alteribacter populi FJAT 45347 (66.7%).

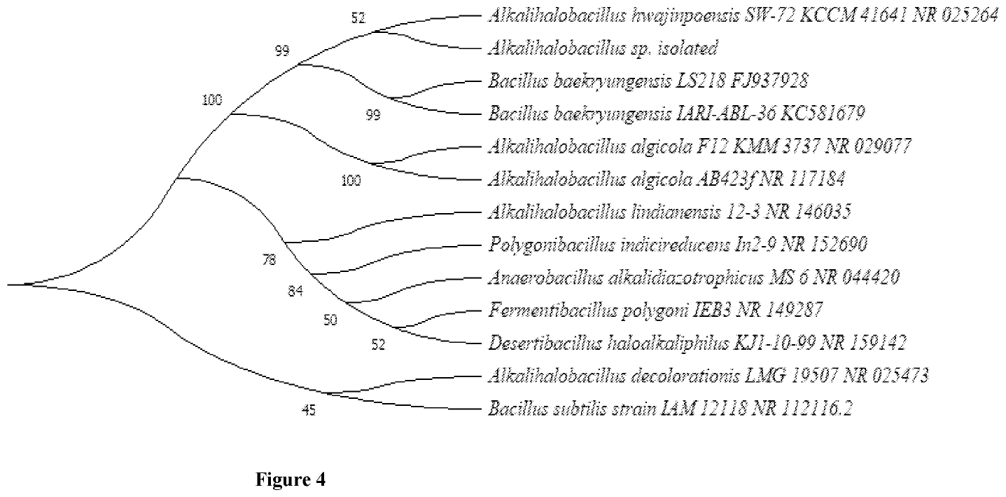

The phylogenetic analysis based on the 16S rRNA gene resulted in high identity of this gene in current isolate to that in the bacterial strain Alkalihalobacillus hwajinpoensis SW-72 (Taxonomy ID: 208199; accession No. AF541966) at a similarity level of >99.5% (FIG. 4).

### Example 2: In Vitro Growth and α-Amylase Activity

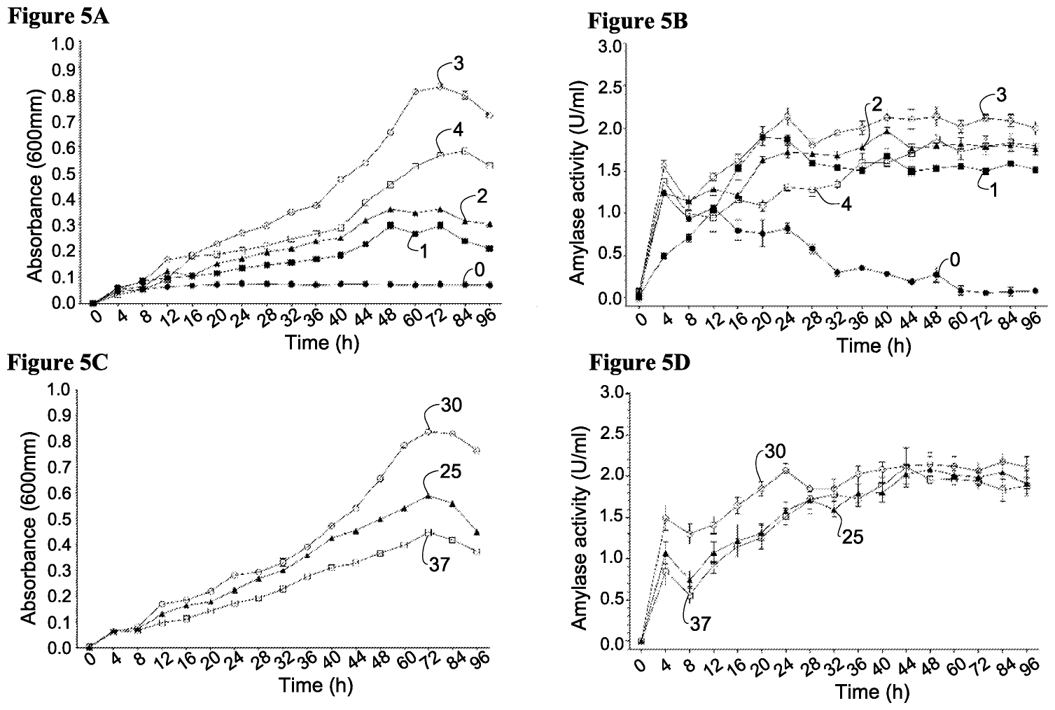

Growth of the isolated Alkalihalobacillus sp. was affected by salinity (p<0.0001) while the increase in culture salinity up to the level of 3% accompanied by greater bacterial yields and faster growth. In the salt-free cultures (0%) bacterial growth was nearly undetectable and bacterial yield, which was the lowest as compared to other cultures, remained nearly similar between 8 to 72 h post inoculation into the medium (FIG. 5A).

The highest yield was measured in cultures at salinity of 3% after 72 h and was between 1.4 and 2.8 times higher than the measured maximal yields in the cultures at other salinity levels (not including the non-saline culture; FIG. 5A). The calculated generation time from fastest to slowest was 16, 20.3, 23.7, or 24 h for cultures at respected salinity level of 3, 4, 2, or 1%.

The effect of temperature on bacterial growth was also significant (p<0001), with greatest bacterial yields and fastest growth at 30° C. At this temperature, maximal yield in the culture medium was identified after 72 h and was 1.5 or 2 times higher than in cultures at 25 or 37° C., respectively (FIG. 5C). Following the measured yields, the fastest generation time was calculated in cultures at 30° C. and was 16.3 h, as compared to 18.1 or 22 h at temperatures of 25° C. or 37° C., respectively.

No α-amylase enzymatic activity was observed in the control cultures in the absence of starch in the medium (not shown). The activity of α-amylase of Alkalihalobacillus sp. was affected by culture salinity (p<0.0001) and resulted in the fastest activity of the enzyme in cultures at salinity of 3% (FIG. 5B). The most significant increase in the activity rate of the enzyme was measured in all of the cultures during the first 4 h after inoculation of the bacterium. In cultures at salinity level of 1, 2, or 3% the activity of the enzyme continued to increase only moderately until reaching maximal rate 24 h post inoculation while at salinity of 4% the maximal activity rate of the enzyme was measured only 48 h after bacterial inoculation. In the non-saline cultures, the maximal activity of the enzyme 4 h after inoculation was followed with a continuous decrease until reaching a nearly non-detectable activity after 60 h from bacterial inoculation (FIG. 5B). The activity of α-amylase in cultures at similar salinity (3%) but different temperatures was relatively similar with the most rapid activity at 30° C. during the first 24 h of inoculation, followed by a relatively similar activity of the enzymes in the different cultures until the end of the in vitro experiment (FIG. 5D).

Table 2 shows statistical analyses of the two-way ANOVA test for the in vitro trial with Alkalihalobacillus sp. examining the effects of different factors of salinity (a, b), or temperature (c, d), as well as of culture time (a, b, c, d), on bacterial growth (a, c) or α-amylase activity (b, d). The impact of integrated factors culture time and salinity or culture time and temperature was also examined and presented under the term interaction.

TABLE 2
statistical analyses of the two-way ANOVA test for the in vitro trial with Alkalihalobacillus sp.

| | | | | | | |
|---|---|---|---|---|---|---|
| Source of Variation | % of total variation | P value | SS | DF | MS | F (DFn, DFd) |

a Optimum salinity for bacterial growth

| | | | | | | |
|---|---|---|---|---|---|---|
| Interaction | 21.78 | <0.0001 | 2.1 | 64 | 0.03281 | F (64, 170) = 942.1 |
| (time + salinity) | | | | | | |
| Time | 44.6 | <0.0001 | 4.299 | 16 | 0.2687 | F (16, 170) = 7716 |
| Salinity | 33.55 | <0.0001 | 3.234 | 4 | 0.8086 | F (4, 170) = 23219 |
| Residual | | | 0.00592 | 170 | 3.482E−05 | |

b Optimum salinity for amylase activity

| | | | | | | |
|---|---|---|---|---|---|---|
| Interaction | 20.83 | <0.0001 | 21.92 | 64 | 0.3425 | F (64, 170) = 30.18 |
| (time + salinity) | | | | | | |
| Time | 29.55 | <0.0001 | 31.09 | 16 | 1.943 | F (16, 170) = 171.2 |
| Salinity | 47.78 | <0.0001 | 50.27 | 4 | 12.57 | F (4, 170) = 1107 |
| Residual | | | 1.929 | 170 | 0.01135 | |

c Optimum temperature for bacterial growth

| | | | | | | |
|---|---|---|---|---|---|---|
| Interaction | 7.779 | <0.0001 | 0.5691 | 32 | 0.01778 | F (32, 102) = 641.0 |
| (time + temperature) | | | | | | |
| Time | 81.91 | <0.0001 | 5.992 | 16 | 0.3745 | F (16, 102) = 13499 |
| Temperature | 10.28 | <0.0001 | 0.7518 | 2 | 0.3759 | F (2, 102) = 13550 |
| Residual | | | 0.00283 | 102 | 2.774E−05 | |

d Optimum temperature for amylase activity

| | | | | | | |
|---|---|---|---|---|---|---|
| Interaction | 3.09 | <0.0001 | 1.556 | 32 | 0.04862 | F (32, 102) = 3.348 |
| (time + temperature) | | | | | | |
| Time | 88.41 | <0.0001 | 44.52 | 16 | 2.782 | F (16, 102) = 191.6 |
| Temperature | 5.556 | <0.0001 | 2.797 | 2 | 1.399 | F (2, 102) = 96.32 |
| Residual | | | 1.481 | 102 | 0.01452 | |

### Example 3: Functional Analysis of the Genome of Alkalihalobacillus Sp.

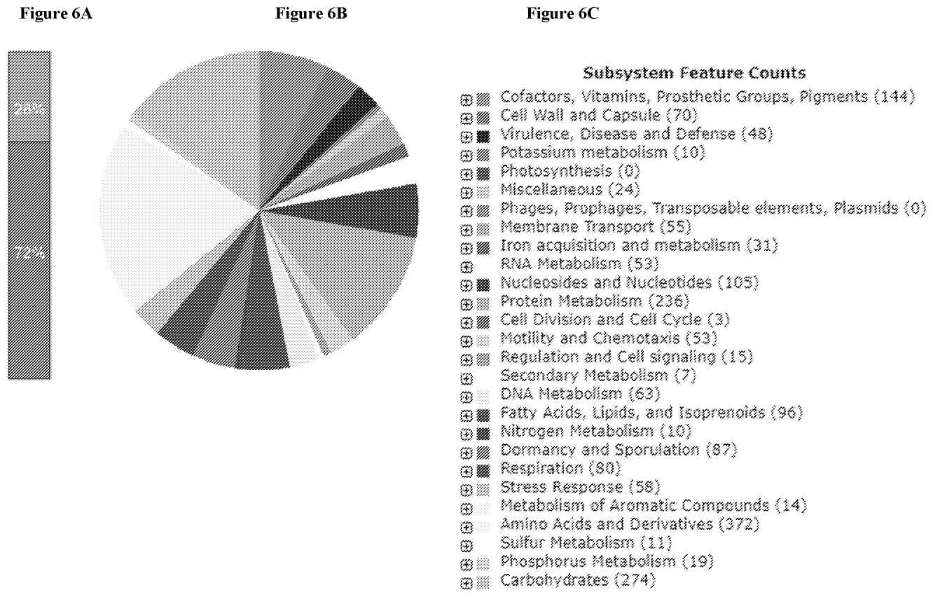

Analysis of the whole genome sequence of Alkalihalobacillus sp. through the RAST server (FIG. 6) resulted in the identification of 4598 CDSs while 1275 of them (28%) were successfully annotated into functional genes which were then clustered into 25 subsystem categories (FIG. 6). Much of the genes were clustered under the metabolism categories of amino acids and derivatives (372), carbohydrates (274), and protein metabolism (236).

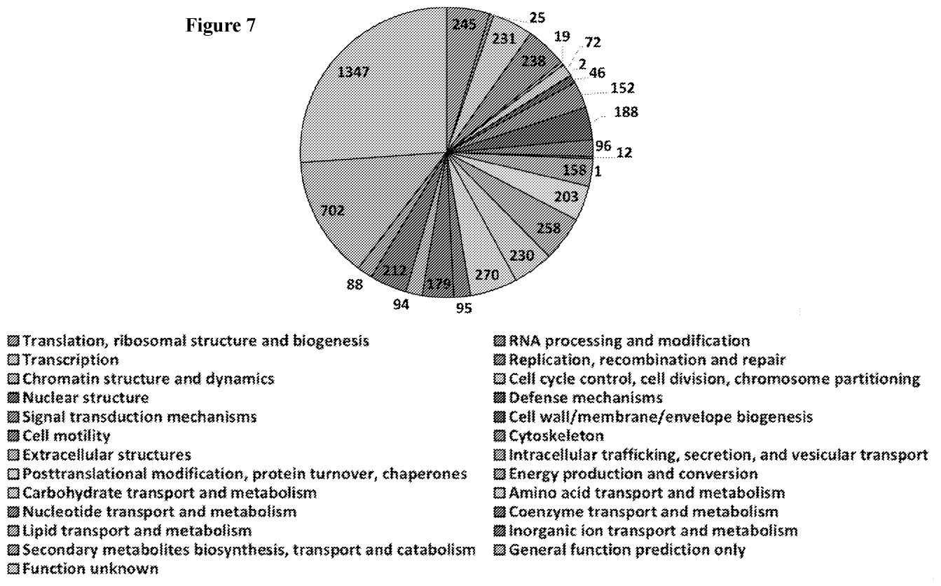

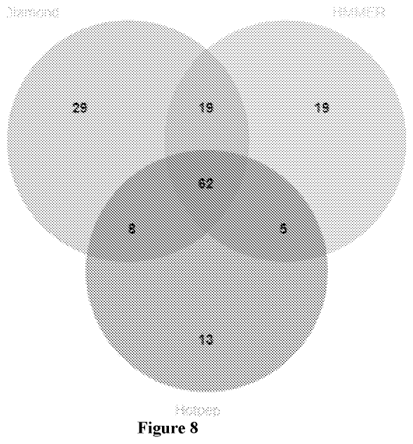

COG analysis revealed 5163 CDSs, of which 3816 (74%) were successfully assigned into known protein coding gene orthologues in the KEGG server while the rest were assigned as genes of either general function or unknown (FIG. 7). 1426 CDSs were assigned as gene orthologues in the category of ‘metabolism’ which was predominated by gene orthologues of the sub-categories of amino acid transport and metabolism (270); energy production (258); and conversion carbohydrate transport and metabolism (230). 758 CDSs were assigned as gene orthologues in the category of ‘information storage and processing’ much of them under the sub-categories of translation, ribosomal structure and biogenesis (245); DNA replication, recombination and repair (238); and transcription (231). Gene orthologues that were assigned under category of ‘cellular process and signaling’ were found mostly under the sub-categories of posttranslational modification, protein turnover and chaperones (203) and cell wall/membrane/envelope biogenesis (188).

### Example 4: The CAZome of Alkalihalobacillus Sp.

Alkalihalobacillus sp. CAZome contained a pool of 94 copies of CAZymes with verified annotations (FIG. 8). CAZymes were clustered in 40 families: 19 GH families, 11 CBM, 6 GT, 3 CE, and an additional single gene of AA (Table 3). Only family GT4 was represented in the genome by the double-digit number of gene copies of 16, followed by CAZymes of another GT family of GT2, and the two families of GHs GH13, and GH32 each with a total number of 9 gene copies. The CBMs in bacterial CAZome included modules for binding of starch (3 families of CBM 20, 34, and 41; Table 3) but also for other substrates as cellulose, glycogen, peptidoglycan, chitin, inulin, levan, and xylan. GHs with an active site for these polysaccharides were also identified, with the highest number of subfamilies and gene copies for those targeting the α-glycoside/α-amylase linkage (GH13, 5 subfamilies). No β-amylases were detected. Among identified CEs, family CE4 had 6 gene copies for enzymes that catalyze de-acylation of polysaccharides.

Table 3 presents the different enzyme families in the genome following their categorization as either glycoside hydrolases (GH); glycosyltransferases (GT); carbohydrate esterases (CE); carbohydrate-binding modules (CBM), or auxiliary activity (AA). The number of copies of each enzyme family as measured in the genome of Alkalihalobacillus sp. is provided together with the functionality of enzymes of this family as identified in the CAZy database.

TABLE 3
List of the various CAZymes genes in the genome of the
here isolated bacterium Alkalihalobacillus sp.

| | Number | |
|---|---|---|
| Enzyme family | of copies | Activities in family |
| AA3_2 | 1 | Glucose-methanol-choline oxidoreductases |
| CBM13 | 1 | Cellulose-binding domain |
| CBM20 | 1 | Starch-binding |
| CBM34 | 2 | Starch-binding |
| CBM38 | 1 | The inulin-binding function and hydrolyze fructose |
| | | containing polysaccharides |
| CBM4 | 1 | Binding of xylan, β-1,3-glucan, β-1,3-1,4-glucan, β-1,6- |
| | | glucan and amorphous cellulose |
| CBM41 | 1 | Starch-binding |
| CBM48 | 4 | Glycogen-binding function |
| CBM50 | 3 | Binding of N-acetylglucosamine residues in bacterial |
| | | peptidoglycans and in chitin |
| CBM6 | 1 | Binding of amorphous cellulose and β-1,4-xylan. |
| | | Some of these modules also bind β-1,3-glucan, β-1,3-1,4- |
| | | glucan, and β-1,4-glucan |
| CBM66 | 1 | Binding of fructose but with higher affinity to inulin and |
| | | levan |
| CBM68 | 1 | Starch-binding |
| CE14 | 2 | N-acetyl-1-D-myo-inosity1-2-amino-2-deoxy-α-D- |
| | | glucopyranoside deacetylase |
| CE4 | 6 | Catalyze the de-acylation of polysaccharides, include |
| | | acetylxylan esterases, chitin deacetylases, |
| | | chitooligosaccharide deacetylases, and peptidoglycan |
| | | deacetylases |
| CE9 | 1 | Catalyze the deacetylation of N-acetylglucosamine-6- |
| | | phosphate to glucosamine-6-phosphate |
| GHI | 3 | Glycoside hydrolases in this family are β-glucosidases and |
| | | β-galactosidases |
| GH13 | 9 | Glycoside hydrolase family acting on substrates containing |
| | | α-glucoside linkages (α-amylase) |
| GH15 | 2 | Glycoside hydrolases of this family are exo-acting enzymes |
| | | that hydrolyze the non-reducing end residues of α- |
| | | glucosides |
| GH16_3 | 1 | Glycoside hydrolases of this family are active on β-1,4 or β- |
| | | 1,3 glycosidic bonds in various glucans and galactans |
| GH18 | 2 | Glycoside hydrolases of this family are both catalytically |
| | | active chitinases |
| GH2 | 2 | Glycoside hydrolases of this family are β-galactosidases, β- |
| | | glucuronidases, β-mannosidases, exo-β-glucosaminidases |
| GH23 | 3 | The glycoside hydrolases of this family are lytic |
| | | transglycosylases |
| | | (also referred to as peptidoglycan lyases) of both bacterial |
| | | and bacteriophage origin |
| GH3 | 1 | The glycoside hydrolases of this family are exo-acting β-D- |
| | | glucosidases, α-L-arabinofuranosidases, |
| | | β-D-xylopyranosidases, N-acetyl-β-D-glucosaminidases |
| | | and N-acetyl-β-D-glucosaminide phosphory lases |
| GH30 | 1 | The glycoside hydrolases of this family are β- |
| | | glucosylceramidase, β-1,6-glucanase, and β-xylosidase |
| GH31 | 1 | The glycoside hydrolases of this family are one of the two |
| | | major families |
| | | of glycoside hydrolases, along with GH13, that contain α- |
| | | glucosidases |
| GH32 | 9 | Glycoside hydrolases of this family are active on fructose |
| | | containing polysaccharides |
| GH35 | 1 | The glycoside hydrolases of this family are β-galactosidases |
| GH36 | 2 | The glycoside hydrolases of this family are α-galactosidase |
| | | and α-N-acetylgalactosaminidase |
| GH38 | 1 | The glycoside hydrolases of this family are Class II α- |
| | | mannosidases |
| GH4 | 1 | The glycoside hydrolases of this family are α-glucosidases, |
| | | α-galactosidases, α-glucuronidases, |
| | | 6-phospho-α-glucosidases, and 6-phospho-β-glucosidases |
| GH42 | 1 | The glycoside hydrolases of this family are β- |
| | | galactosidases. |
| GH52 | 1 | The glycoside hydrolases of this family are exo-β- |
| | | xylosidases |
| GH68 | 2 | The glycoside hydrolases of this family are levansucrase, β- |
| | | fructofuranosidase and inulosucrase |
| GH73 | 1 | The glycoside hydrolases of this family are enzymes cleave |
| | | the β-1,4-glycosidic linkage between N-acetylglucosaminyl |
| | | and N-acetylmuramyl (NAM) moieties in the carbohydrate |
| | | backbone of bacterial peptidoglycans |
| GT0 | 2 | These enzymes catalyse the transfer of sugar moieties from |
| | | activated donor molecules |
| | | to specific acceptor molecules and forming glycosidic |
| | | bonds |
| GT2 | 9 | Proteins in this entry contain glycosyl transferase family 2 |
| | | domains which are responsible, |
| | | generally, for the transfer of nucleotide-diphosphate sugars |
| | | to substrates such as polysaccharides and lipids |
| GT28 | 1 | Glycosyltransferase with a number of known activities; 1,2- |
| | | diacylglycerol 3-beta-galactosyltransferase; |
| | | 1,2-diacylglycerol 3-beta-glucosyltransferase; beta-N- |
| | | acetylglucosamine transferase |
| GT4 | 16 | Sucrose synthase bacterial phospho-N-acetylmuramoyl- |
| | | pentapeptide-transferases, |
| | | which catalyse the first step of the lipid cycle reactions in |
| | | the biosynthesis of cell wall peptidoglycan |
| GT5 | 1 | This enzyme involved in the pathway starch biosynthesis, |
| | | which is part of glycan biosynthesis |
| GT51 | 5 | Murein polymerase enzyme utilise MurNAc-GlcNAc-P-P- |
| | | lipid II as the sugar donor |

### Example 5: Comparison of the CAZome of Alkalihalobacillus Sp. To Other Selected Bacteria

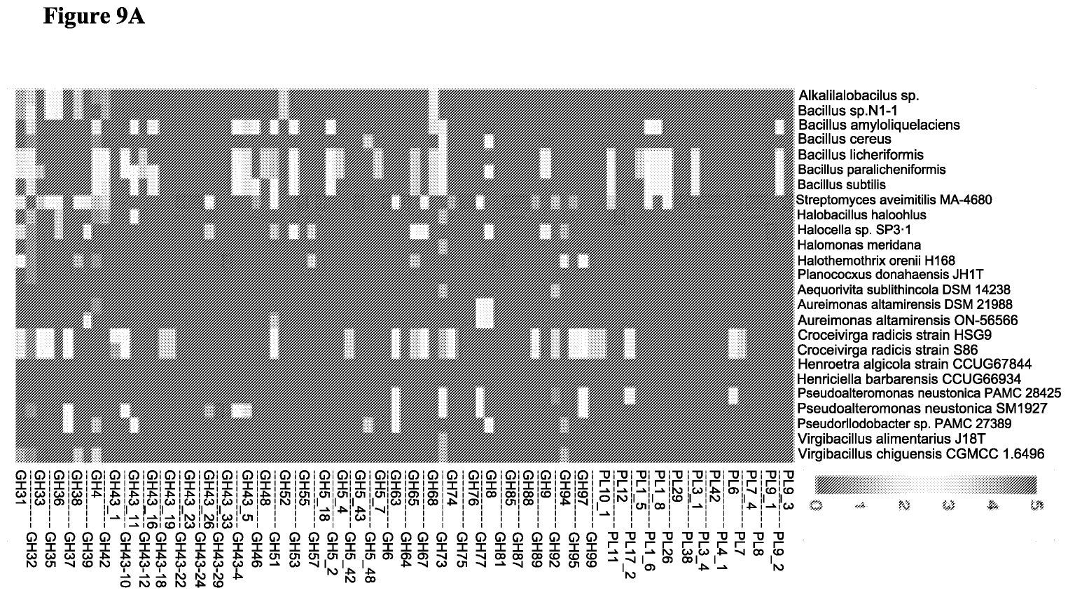

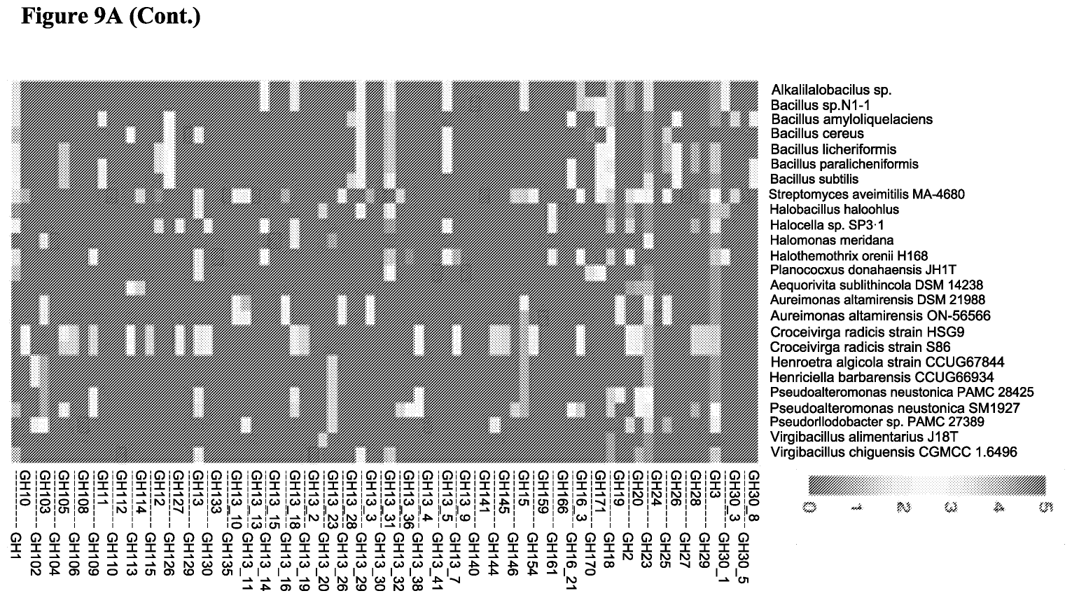

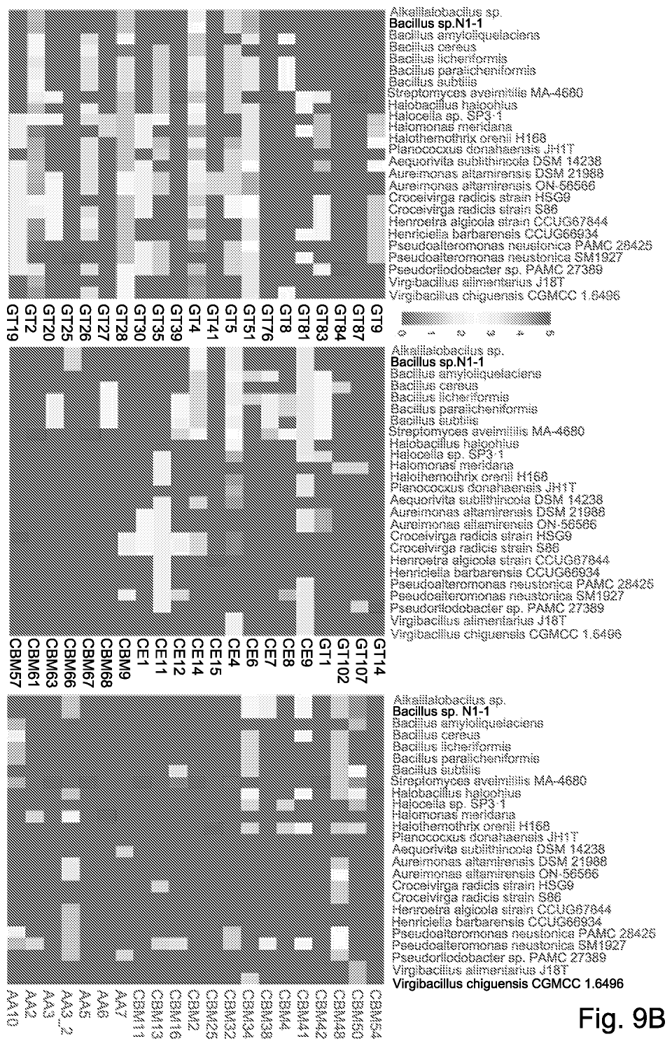

A comparative analysis of the CAZomes of Alkalihalobacillus sp. isolate and other selected bacteria revealed a total number of 224 CAZymes with 140 GHs, 25 GTs, 21 PLs, 21 CBMs, 10 CEs, and 7 AAs (Table 3) (FIGS. 9A and 9B). The CAZome of halophilic bacterium Streptomyces avermitilis MA-4860 was richest and contained 105 CAZymes, followed by the two strains of halophile Croceivirga radicis that consisted of 68 or 67 CAZymes. S. avermitilis MA-4860 also presented 38 unique CAZymes, many of them in numerous copies (>5). The poorest CAZome was in bacterium Virgibacillus alimentarius J18T and contained only 11 CAZymes. Among the commercially important Bacillus, B. paralicheniformis and B. cereus were richest (66) or poorest (34) in CAZymes, respectively. Only CAZymes GT28 and GT51 were present in all examined bacteria, while GTs 2 and 4 were missing only in Pseudoalteromonas neustonica (SM1927). GH23 was also present in nearly all bacteria excluding Halothermothrix orenii H168, while GH3 was not found in Bacillus cereus and Virgibacillus Alimentarius J186.

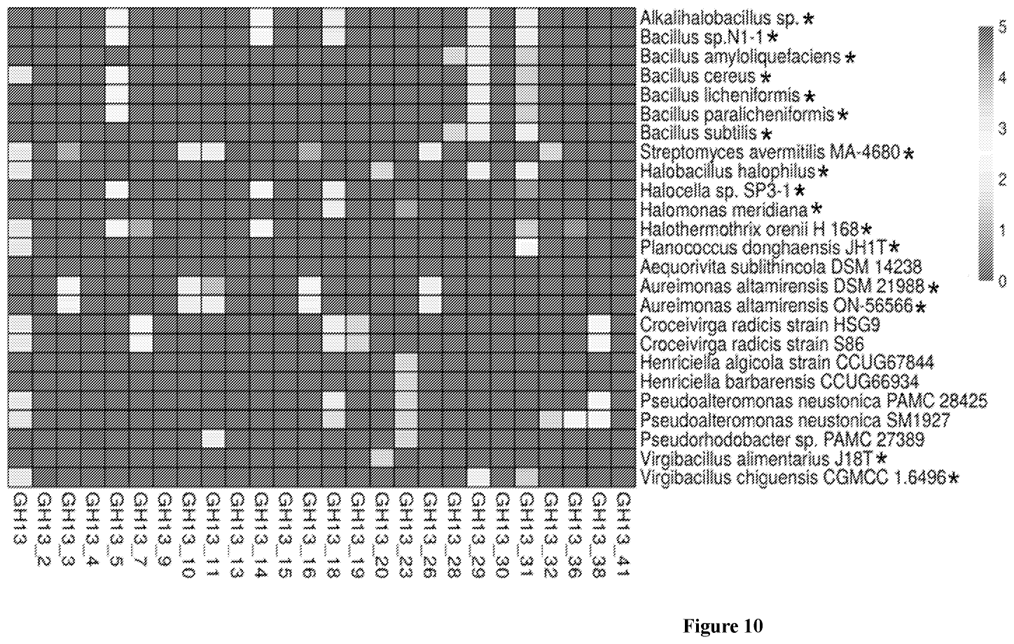

The CAZome of isolated Alkalihalobacillus sp. was most similar to that in the halophile Bacillus sp. N1-1 (FIG. 10) and shared a pool of 36 CAZyme sub-families. 21 of these were GHs, 6 CBMs, 5 GTs, 3 CEs, and 1 AA. Differences between these bacteria included a single GH4 gene in the CAZome of Alkalihalobacillus sp. and 3 unique GHs of 140, 170, and 171 in Bacillus sp. N1-1. The CAZyme families GH13 and GH43 were richest in terms of their content of genes from different subfamilies, with 26 and 15 subfamilies, respectively. Despite this richness in family GH43, 8 of the compared microbes lack GH43 genes while B. paralicheniformis and S. avermitilis each contained 8 CAZymes of this family. A closer examination of the α-amylases (GH13) revealed that only Aequorivita sublithincola DSM 14238 lack CAZymes of this family and Henriciella algicola CCUG67844, Henriciella barbarensis CCUG66934, and Virgibacillus alimentarius J18T had only a single copy of GH13 each. In contrast, S. avermitilis MA-4860 was also richest in α-amylases with 9 GH13 subfamilies, 2 of them unique to this bacterium. The most common GH13 CAZyme was GH13_31 of oligo 1,6-glucosidase, found in 12 of the examined bacteria. The isolated Alkalihalobacillus sp. and Bacillus sp. N1-1 were identical in their α-amylase content, each of these CAZymes appearing in an identical number of copies (FIG. 10). The GH13 CAZome of these two bacteria consisted of a relatively unique set of two α-amylases, GH13_14 and 18, as both together were found only in Halocella sp. SP 3-1.

### Example 6: Starch and Sucrose Metabolism by Alkalihalobacillus Sp. Following CAZome Content

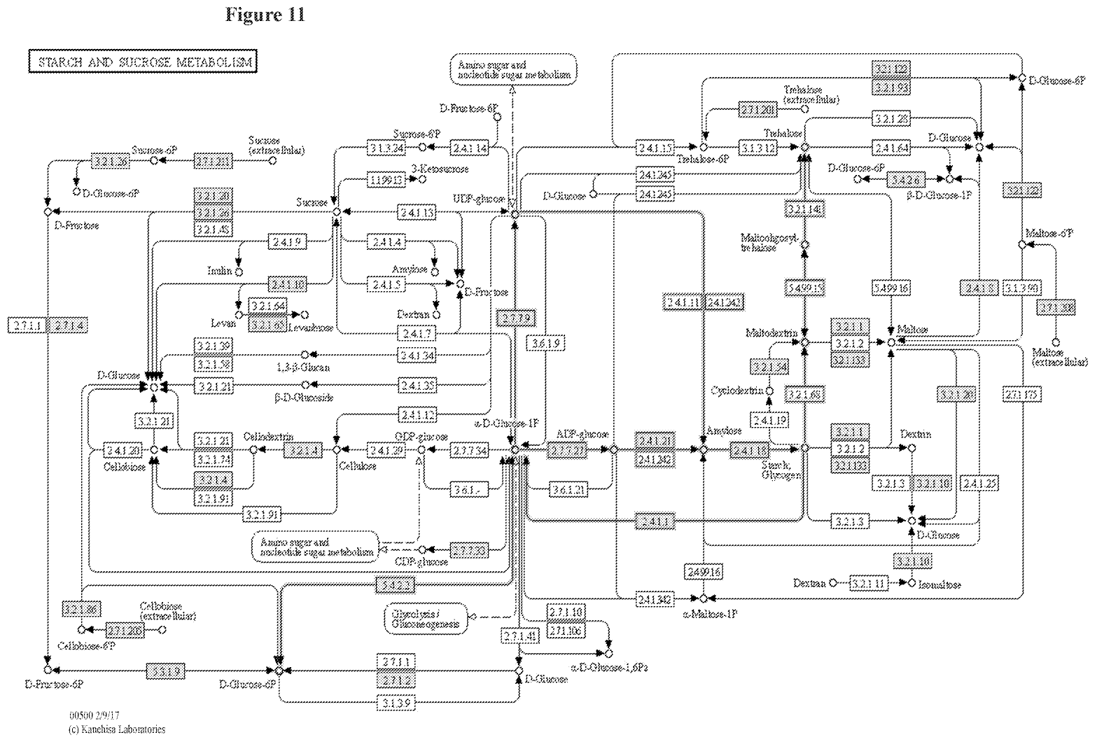

Alkalihalobacillus sp. CAZome revealed two catabolic pathways for degradation of the starch substrate (FIG. 11).

In the first pathway, glycogen phosphorylase (GH13_18, EC:2.4.1.1) break the linear chains in the glycogen substrate by phosphorolytic cleavages of the α-1,4-glucosidic bonds into α-D-glucose-1-phosphate oligo or mono-saccharides. In the following step, UTP-glucose-1-phosphate uridylyltransferase (GH13_29, EC:2.7.7.9) phosphorylate the α-D-glucose-1-phosphate to form another activated form of nucleotide glucose of a UDP-glucose, which is further utilized into metabolic cycles of amino sugars and nucleotide sugars.

The second metabolic pathway involve hydrolysis of starch into dextrin and maltose and their further conversion into D-glucose/dextrose units. The hydrolysis of starch is initiated by the attack of the α-1,4 glycoside bonds by α-amylase of either glucan (GH13_5, EC:3.2.1.1) or glucan 1,4-alpha-maltohydrolase (GH13_14, EC3.2.1.133). In both cases, the hydrolysis of the starch substrate results in the formation of dextrin polymer that is made of D-glucose units and maltose disaccharide made of glucose units. The dextrin polymer is further hydrolyzed into the D-glucose units by oligo-1,6-glucosidase (GH13_31, EC:3.2.1.10) which hydrolyze α-1,6 linkage in the polymer. In the alternative pathway through maltose, this substrate is hydrolyzed into D-glucose by maltose phosphorylase (GH31, EC:2.4.1.8).

1. A marine-derived halotolerant bacterium capable of hydrolyzing at least one carbohydrate.

2. A microbial consortium comprising at least one marine-derived halotolerant bacterium of claim 1 or any isolate, mutant, spores, enzymes or extracts thereof and/or a conditioned culture medium of the at least one bacterium and/or secreted compounds from the at least one bacterium.

3. The bacterium of claim 1, wherein said bacterium is present at a low abundance in intestine of a marine urchin.

4. The bacterium of claim 3, wherein said bacterium is present in Tripneustes gratilla elatensis.

5. The bacterium of claim 1, wherein said bacterium is capable of (i) growing in a solution comprising about 3% saline, (ii) growing at 30° C. or (iii) growing at 30° C. in a solution comprising about 3% saline.

6.-7. (canceled)

8. The bacterium of claim 1, wherein the carbohydrate is (A) a polysaccharide, optionally wherein said polysaccharide is at least one of (i) cellulose, (ii) starch, (iii) glycogen or (iv) any combination thereof, (B) at least one of amylose and amylopectin, (C) from an algal source, (D) an algal polysaccharide, (E) an algal extract, (F) an algal biomass, or (G) one or more of carrageenan, agar, cellulose, alginate, laminarin, fucoidan, ulvan, chitin, starch, xylan, rhamnan sulfate, chrysolaminarin, or any combination thereof.

9.-15. (canceled)

16. The bacterium of claim 1, wherein said bacterium expresses genes capable of metabolizing (i) amino acid and derivatives, (ii) carbohydrate, (iii) protein metabolism or (iv) a combination thereof.

17. The bacterium of claim 1, wherein said bacterium (A) express gene copies of at least one carbohydrate active enzyme (CAZome), optionally wherein said bacterium comprises at least 80 gene copies, optionally at least 90 gene copies of CAZymes, (B) contain at least one carbohydrate active enzyme (CAZome), or (C) produces at least one carbohydrate active enzyme (CAZome).

18.-20. (canceled)

21. The bacterium of claim 17, wherein said CAZome comprise at least one glycoside hydrolase (GH) enzyme, at least one glycosyltransferase (GT) enzyme, at least one polysaccharide lyases (PLs) enzyme, at least one carbohydrate esterases (CEs) enzyme, at least one carbohydrate-binding modules (CBM) enzyme, at least one auxiliary activities (AAs) enzyme or any combination thereof.

22. The bacterium of claim 21, wherein the at least one bacterium contains gene copies of at least one CE enzyme, at least one CBM enzyme, at least one AA enzyme, at least one PL enzyme, at least one GT enzyme, or at least one GH enzyme.

23.-26. (canceled)

27. The bacterium of claim 22, wherein the at least one GT enzyme is at least one of (i) GT4, (ii) GT2, (iii) a combination thereof, optionally (a) wherein said bacterium contain at least 12 GT4 gene copies, optionally at least 15 GT4 gene copies or (b) wherein said bacterium contain at least 5 GT2 gene copies, optionally at least 8 GT2 gene copies.

28.-30. (canceled)

31. The bacterium of claim 27, wherein said GH enzyme is GH4 or at least one of (i) GH13, (ii) GH32, (iii) GH31, (iv) GH16 or (v) a combination thereof.

32. (canceled)

33. The bacterium of claim 31, wherein said bacterium contain at least 6 GH13 gene copies, optionally at least 8 GH13 gene copies, optionally wherein the GH13 is at least one of GH13_31, GH13_29, GH13_18, GH13_14, GH13_5 or a combination thereof.

34. (canceled)

35. The bacterium of claim 31, wherein said bacterium contain at least 6 GH32 gene copies, optionally at least 8 GH32 gene copies.

36. The bacterium of claim 1, wherein said bacterium contain at least one enzyme listed in Table 3.

37. The bacterium of claim 1, wherein said bacterium is capable of hydrolyzing a carbohydrate into at least one of (i) amino sugars and nucleotide sugars, (ii) D-glucose, (iii) D-glucose-6P or (iv) combination thereof.

38. The bacterium of claim 1, wherein said bacterium has at least 85% identity with SEQ ID NO:1, from the genus Alkalihalobacillus; has at least 85% identity with SEQ ID NO:2; or has a sequence as provided in SEQ ID NO:2.

39.-41. (canceled)

42. A bacterial culture comprising a biomass composition and a bacterium of claim 1, optionally wherein said biomass is an algal biomass.

43. (canceled)

44. A method of hydrolyzing carbohydrate, the method comprising contacting a biomass, biomass derivatives or compositions comprising biomass or any derivative thereof with a bacterium of claim 1 with a biomass, optionally wherein said biomass is an algal biomass or any extract thereof.

45. (canceled)

46. A method of production of bioenergy products or metabolites comprising contacting a biomass or biomass derivatives with the bacterium of claim 1, optionally wherein the biomass is an organic matter or an algal biomass.

47.-50. (canceled)
